(19) United States
(12) Patent Application Publication
(10) Pub. No.: US 2013/0189723 A1
(43) Pub. Date: Jul. 25, 2013

(54) AUTOMATED CELL CULTURE SYSTEM AND PROCESS

(71) Applicant: Global Cell Solutions, LLC, Charlottesville, VA (US)

(72) Inventors: Robin A. FELDER, Charlottesville, VA (US); John J. Gildea, Charlottesville, VA (US)

(73) Assignee: Global Cell Solutions, LLC, Charlottesville, VA (US)

(21) Appl. No.: 13/627,520

(22) Filed: Sep. 26, 2012

Related U.S. Application Data
(63) Continuation of application No. 10/893,569, filed on Jul. 19, 2004, now abandoned.
(60) Provisional application No. 60/488,068, filed on Jul. 17, 2003.

(51) Int. Cl. C12N 5/00; C12Q 1/02; C12M 1/00; C12M 1/12

(52) U.S. Cl. CPC C12N 5/0075; C12M 25/14; C12Q 1/02; C12M 23/14
USPC 435/29; 435/397; 435/396; 435/410; 435/366; 435/289.1; 435/287.7; 435/383; 435/374; 435/394

(57) ABSTRACT

The present invention relates generally to the field of cell culture, which is a laboratory process used primarily for the growth, propagation, and production of cells for analysis and the production and harvesting of cell products. The present invention comprises functionalized and/or engineered hydrogel microcarriers that exhibit any or all of the following properties: controllable buoyancy, ferro- or paramagnetism, molecular or fabricated reporting elements, and optical clarity. The microcarriers are used in a bioreactor that employs external forces to control said microcarrier kinetic energy and translational or positional orientation in order to facilitate cell growth and/or cellular analysis. The bioreactor can be part of an automated system that employs any or all of the following; a microcarrier manufacturing method, a monitoring method, a cell culture method, and an analytical method. Either a single bioreactor or a plurality of bioreactors are used in the automated system to enable cell culture and analysis with a minimum of human intervention.

### CROSS-REFERENCE TO RELATED PATENT APPLICATIONS

This application is continuation of U.S. patent application Ser. No. 10/893,569, filed Jul. 19, 2004, which claims priority to U.S. Provisional Application 60/488,068, filed Jul. 17, 2003, incorporated herein by reference in its entirety.

### BACKGROUND OF THE INVENTION

The present invention relates generally to the field of cell culture, which is a laboratory process used primarily for the growth, propagation, and production of cells for analysis and the production and harvesting of cell products. Living cells are usually seeded onto a plastic surface in a growth media containing many of the nutrients and growth factors present in their natural environment. The cells, sitting on the bottom of a plastic vessel, such as a Petri dish or a flask, are then placed into an incubator which provides a warm, moist, and appropriately gassed environment to grow. There is virtually no limit to the number and variety of cells that can be cultured, and valuable products and data that can be obtained from cells in culture. Cultured cells can be used to screen large medicinal compound libraries for potential pharmaceutical activity, and secreted proteins and nucleic acids from cultured cells may have significant value as pharmaceutical products. In addition, cell culture has a wide range of laboratory research applications, such as drug discovery programs in pharmaceutical laboratories, and human, animal and plant cells for cell based therapeutics.

The bulk of traditional cell culture depends on the use of flat bottom dishes on which cells of interest are grown. Petri dishes, and other cell culture ware, provide a surface on which anchorage dependent cells can attach and grow. A traditional Petri dish has a surface area of 78.5² cm and can support the growth of over 1×10⁶ cells when fully confluent. Improvements on the Petri dish have included the use of cell flasks, roller bottles, and growing cells on fibers in culture vessels.

Microcarriers have been developed as an alternative to growing cells on the surface of the growth media container or culture vessel. Microcarriers have been created out of a variety of materials such as plastic, glass, gelatin and calcium-alginate (2, 3, 4, 5), in order to increase the surface area available on which cells can grow. However, microcarriers must be stirred in order to grow cells on their surface. Prior art describes a spinner flask requiring a suspended impeller driven by an external rotating magnet under the base of the spinner flask to maintain the microcarriers in suspension. However, impellers impart hydrodynamic stress on growing cells (6) that can damage cells or alter their morphology. Impellers are usually suspended in the cell culture media and are stirred via a direct coupling to an overhead motor, or through magnetic induction from a rotating magnet in the base of the support for the culture flask. Impellers can be expensive since they have to be made out of material that can be cleaned and sterilized and do not impart any contaminating substances in the cell culture media.

Additionally, the majority of laboratories perform conventional cell culture manually that includes thawing cells from the freezer, seeding them in a culture vessel or flask, growing, feeding and splitting them to eventually scrape or detach them with enzymes for assay and freezing away if necessary.

Thus, there is a need to improve conventional cell culture regarding the handling of the cells during the culturing, maintenance and analysis of the cells and to improve the status or health of the cells in culture and the conditions in which the cells are grown so in some cases the cells are grown in an environment more like the environment in which the cells are grown in nature. This improvement in growth conditions will provide more accurate analyses and observation because the cell culture conditions will mimic or be a more accurate representation of the physiological conditions of cell in the organism from which it originally was obtained, such as humans, non-human mammals, animals, plants, and others. In terms of reduction in manipulative steps, in some embodiments, the present invention can reduce the labor required to handle the cells by approximately 75% to eliminate the traditional manipulative steps of seeding, growing, feeding, splitting and assaying the cells or cell products.

### SUMMARY OF THE INVENTION

The present invention is directed to an engineered microcarrier suitable for growing cells comprising a hydrogel composition capable of providing a substrate that will support the growth of cells in culture, wherein said gel composition further comprises at least one material which renders the microcarrier responsive to at least one physical force. The cells may grow inside of and outside on the surface of the engineered microcarriers, which have been produced to respond to, to be manipulated by or to be controlled by at least one physical force when used in a cell culture system. The present invention also is directed to methods of making these engineered microcarriers and methods of use to grow cells for analysis and production of cell products.

In another embodiment, the present invention further is directed to a bioreactor comprising the engineered microcarriers as described herein contained in a culture vessel or bioreactor and a source for emitting a force into, around and/or outside of the culture vessel that will control the movement of the engineered microcarriers within the culture vessel, wherein the source is controlled by a In a further embodiment, the present invention additionally is directed to an automated cell culture system comprising the engineered microcarriers, a culture vessel or bioreactor and a source for emitting a force into, around and/or outside of the culture vessel that will control the movement of the engineered microcarriers within the culture vessel, wherein the source is controlled by a control system. and bioreactors to achieve the goals of culturing cells.

One embodiment of the invention relates to an automated cell culture system and monitoring system comprising the engineered microcarriers, a culture vessel or bioreactor and a source for emitting a force into, around and/or outside of the culture vessel that will control the movement of the engineered microcarriers within the culture vessel, wherein the source is controlled by an integrated control system and further comprising a monitoring system that view, measures, records, and transmits data to an integrated computer processor or biochip processor which controls the process.

### BRIEF DESCRIPTION OF THE DRAWINGS

FIG. 1 is a representation of a conventional microsphere, a microsphere with paramagnetic particles, a microsphere with buoyant elements and paramagnetic particles.

FIG. 2 is a diagram of a representative bioreactor of the present invention containing engineered microcarriers of the present invention showing the relationship of the source of applied physical force to the culture vessel and an opening for the addition and removal of media and/or microcarriers.

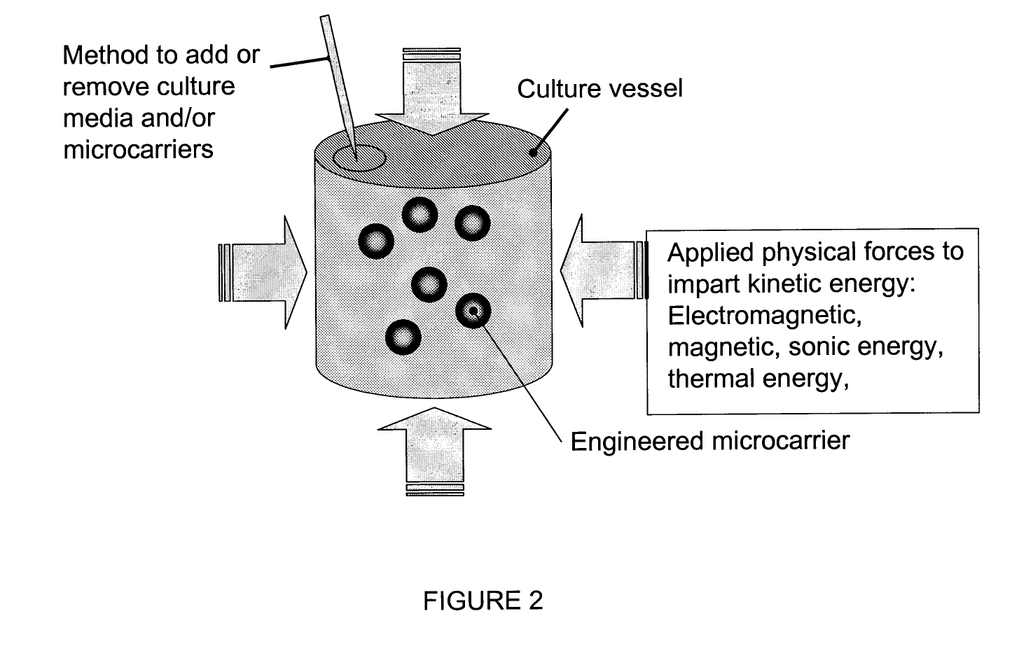

FIG. 3 is a diagram of a representative automated bioreactor of the present invention containing engineered microcarriers of the present invention which is controlled by a control system that also controls the source of the physical force, the addition or removal of the media and microcarriers from the culture vessel, through an opening and the monitoring system.

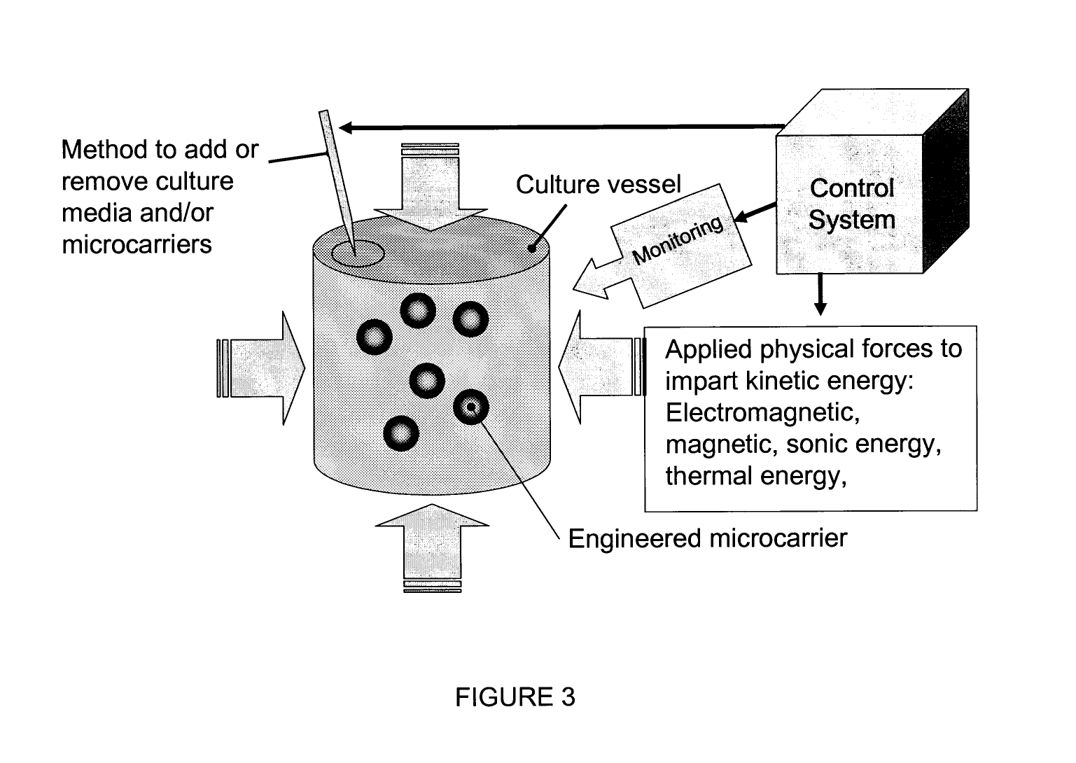

FIG. 4 is a diagram of a representative automated bioreactor of the present invention containing engineered microcarriers and further showing the relationship to a microcarrier manufacturing method from which microcarriers are provided directly into the culture vessel and its relationship to an assay method which receives microcarriers from the culture vessel for analysis. The control system controls the automated culture vessel system in the boxed area as well as the microcarrier manufacturing method and the assay method.

FIG. 5 is a representation of one embodiment of the present invention that utilizes a single magnet. The figure shows how this magnet is used to move the microcarriers within the bioreactors. Two bioreactors comprising a culture vessel and a source of a physical force, a single electro- or permanent magnet for each culture vessel are shown. In the left figure, the magnets represented by the dark disc are moved down to the bottom of the culture vessel to pull the microcarriers represented by small circles to pull off waste media through the opening on the right side of the culture vessel. In the right, the magnets are moved down to the top of the culture vessel to pull the microcarriers represented by small circles to pull off microcarriers from the culture vessel.

FIG. 6 is a representation of an embodiment of the present invention when a series of electromagnetic coils or magnets are used to encircle a culture vessel. This representation shows that microcarriers can be moved according to their cell growth needs and to facilitate media changing and microcarrier aspiration. The top coil is energized to move microcarriers up for aspiration manually or by a robot arm. All coils can be energized to keep the microcarriers in suspension. The bottom coil is energized to move microcarriers to the bottom for removal of waste media and addition of fresh media.

FIG. 7 is a representation of media change in the left figure and microcarrier aspiration in the right figure. This figure shows a similar use of the magnets as FIG. 5 but with a plurality of the magnets as in FIG. 6.

FIG. 8 is a representation of an alternative magnet arrangement that will allow microcarriers to be manipulated according to specific needs. As in FIG. 6, the top magnet coil moves the microcarriers up for manual or robotic aspiration of microcarriers, the bottom magnet coil moves the microcarriers down for manual or robotic aspiration of used or waste media and all of the coils are oscillated to keep the microcarriers in suspension.

FIG. 9 is a further representation of magnetic fields to provide a variety of microcarrier movements. This figure demonstrates the use of two circular electromagnets with two poles or multiple poles to effect diagonal movement through the culture vessel.

FIG. 10 is a representation of a further alternative arrangement of magnets allowing more circular and top to bottom mixing of the microcarriers.

FIG. 11 shows a representation of an engineered microcarrier that is manipulated by external magnetic fields to induce kinetic energy. The microcarrier is rotated on its axis to induce shear stress on cells growing on the exterior of the sphere and cells around the perimeter are expected to be exposed to greater shear stresses as compared to those near the axis as approximated in the Shear Force Profile to the right of the sphere.

### DETAILED DESCRIPTION OF THE PREFERRED EMBODIMENTS

The present invention discloses microcarriers that have been modified from the conventional microcarriers to also contain additives that provide specific properties that result in the manipulation and physical movement of the microcarriers in relation to other microcarriers or simply movement within the culture vessel. The present invention further discloses microcarriers in which the additives are ligands, reporters or response elements that report a stimulus or respond to a stimulus.

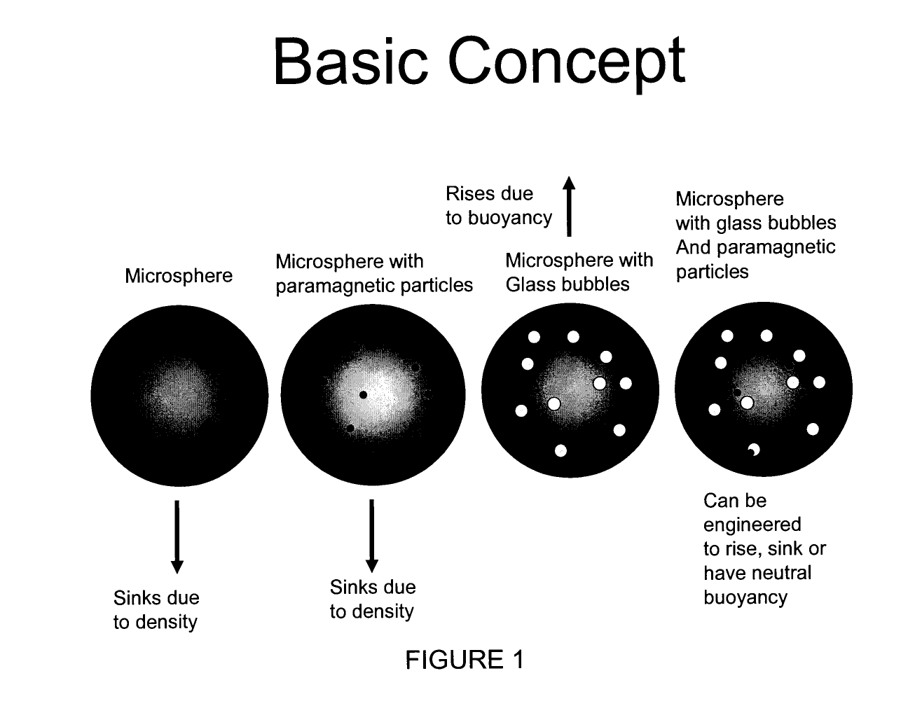

The present invention further discloses engineered microcarriers that are made with a wide variety of substances with virtually unlimited properties. For example, such engineered microcarriers include but are not limited to gelatin, polyacrylamide-copolymerized with collagen or gelatin, polyacrylamide with modified charge, alginate, and alginate copolymerized with gelatin. A preferred microcarrier is one made with a chemical format, such as calcium-alginate and gelatin, as disclosed in Kwon et al. (7). But these conventional microcarriers are then modified to produce functionalized microcarriers that act as reporters. We also describe improvements over chemical microcarriers that are engineered microcarriers possessing specific properties, such as specific buoyant and magnetic and/or paramagnetic properties, as descried herein. Thus, the functionalized and/or engineered microcarriers of the present invention comprise properties of known microcarriers in that they are produced from chemical compounds and compositions using known methods and materials but these conventional microcarriers are further engineered or modified to contain or comprise additives that provide these advantageous properties, such as particles, molecules and/or gases, introduced into the microcarrier (See FIG. 1) or alternatively attached to the outside of the microcarrier that impart changes in density and/or allow the engineered microcarrier to be moved, steered, agitated or otherwise manipulated around the inside of a culture vessel or bioreactor by at least one applied physical force that imparts kinetic energy to the engineered microcarriers inside the culture vessel.

The calcium alginate and gelatin microcarriers are particularly useful for monitoring cell function since the resulting engineered microcarriers made from these compositions have minimal endogenous fluorescence allowing the cells to be observed using microscopic techniques, such as fluorescence confocal microscopy. A preferred embodiment is the creation of microcarriers that are optically clear when compared to other microcarriers that are available. Preferred microcarriers disclosed in the present invention retain a large proportion of their optical clarity, and functionally do not interfere with observations or quantitative measurements that are carried out on the cells inside or outside of the engineered microcarriers, even when engineered with additives as described in more detail herein.

The microcarriers of the present invention provide for an increase in cellular density, for example, cancer cells grow to a density of up to 7×10⁵ cells for every 5×10³ microcarriers. However, when cells have grown to a sufficient density, as determined by reporter molecules integral to the microcarriers indicate the degree of confluence, or the use of an external monitoring system, they are used directly for studies since they contain reporter molecules without the need for the relatively disruptive process of releasing them from attachment using enzymes such as trypsin (as is necessary in flat bottom vessels).

The present microcarriers convey the benefit of flexibility in that they can be created with unlimited geometric, chemical, and functional properties (8, 9). In some embodiments of the present invention, microcarriers may be made as spheres of any diameter, but more reasonably in a range from 1 mm down to less than the diameter of the cell of interest. Microcarriers of the present invention preferably range in size from 1 μm up to 1 mm in diameter. However, smaller sizes down to less than one nanometer and larger sizes up to 2 centimeters or more are microcarriers that are useful in the disclosed automated cell culture system and are produced by the disclosed techniques. Microcarriers useful in the present invention may also be chemically modified to allow non-adherent cells to attach to their surface. The present invention in some embodiments employs a technology that allows non-adherent cells to attach (10), but allows such attachment to the further engineered microcarriers possessing specific reporting, buoyant and magnetic and/or paramagnetic properties as descried herein.

The engineered microcarriers of the present invention are useful generally to facilitate harvesting operations; however, the tendency for microcarriers to settle out of suspension does not allow them to be easily harvested by automation. Microcarrier products have been on the market for several decades, but interest in their use to support the high throughput screening process in the pharmaceutical industry has been stymied by their difficulty to manipulate and the expensive and complicated impeller systems or growth vessel rotation systems needed to use them. The use of engineered microcarriers of the present invention in an automated cell culture system and monitoring system as disclosed herein for high throughput screening provides advantages over previously used high throughput screening systems.

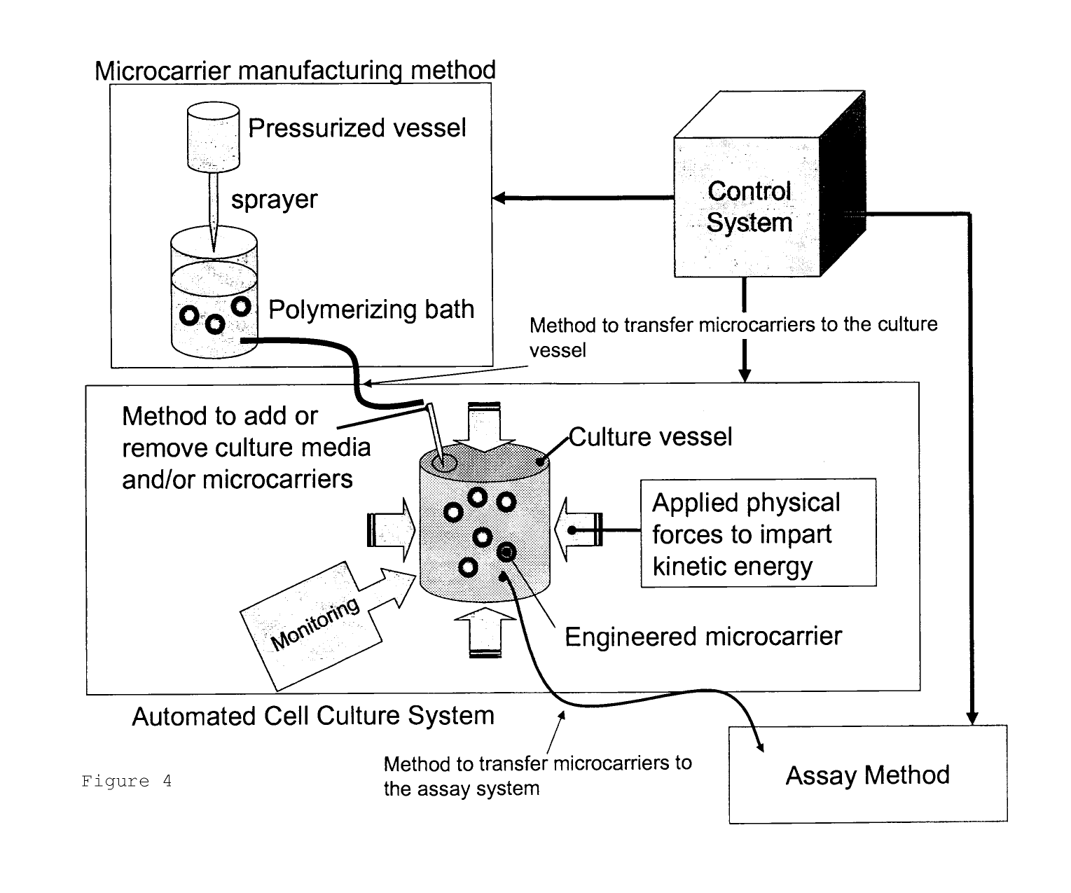

In another embodiment, the present invention discloses manufacturing or producing microcarriers using conventional techniques, including spraying into a liquid containing a polymerizing chemical mixture, or by adding the microcarrier matrix to a rapidly stirring oil bath in order to create an emulsion. In another embodiment of the present invention, the manufacturing of the engineered microcarriers may optionally be an integrated process within the cell culture automation platform (see FIG. 4). To date, a cell culture system does not exist that manufactures the needed engineered microcarriers as they are needed for use in the automated cell culture system as disclosed herein. This approach provides a “just in time” cell culture production process.

### Engineered Microcarriers

### Magnetism

The novel engineered microcarriers of the present invention comprise incorporated buoyant elements that effect the density of the engineered microcarriers and/or particles that impart magnetic and/or paramagnetic properties to the engineered microcarriers. The use and incorporation of miniature magnetic or paramagnetic particles allows the control of the particles by external magnetic fields. The choice of magnetic and/or paramagnetic particles allows one to reduce the size or orientation of the external magnetic field necessary to impart selected movement or kinetic energy on the engineered microcarriers. The benefit of incorporating paramagnetic particles in the engineered microcarrier is their lack of inherent magnetism when they are not being exposed to an external magnetic field, which would then prevent their attraction to each other. In some embodiments, microcarrier aggregation is desirable when creating useful aggregates of cells, such as building tissues or organs. Thus, microcarriers may be induced to aggregate in specific orientations and numbers by any combination of internal magnetic or ferromagnetic properties coupled with any arrangement of external magnets (either permanent or electromagnet). In other embodiments aggregation is undesirable, such as in high throughput screening for novel pharmaceuticals where discrete microcarriers may yield higher screening signals. In further embodiments, a combination of paramagnetic and magnetic material is desirable to impart properties that allow variable response to an external magnetic field.

Magnetic properties of the engineered microcarriers may be controlled during the manufacturing of these microcarriers, or imparted after the manufacturing process. If microcarriers are polymerized or gelled in the absence of a magnetic field, then the magnetic or paramagnetic particles will have a random orientation on or within the engineered microcarrier. On the other hand, if a magnetic field is applied in a static or varying way during the manufacturing process, then one can impart a specific orientation and magnetic strength (if the particles can be magnetized) to the particles on or within the microcarrier. For example, but not meant to limit the present invention, one might wish to impart a magnetic dipole to each microcarrier so that they may be rotated on their axis as the result of exposing the microcarriers in a liquid to an external magnetic field. If the user does not desire microcarrier aggregation as a result of cells growing on their surface sticking to each other, then imparting an axial rotation would tend to prevent inter-microcarrier aggregation.

### Buoyancy

The buoyancy of the microcarriers is controlled by either manufacturing them out of materials with buoyant properties, or by adding a substance or substances which can control buoyancy. Buoyancy is defined herein as the property that will make the microcarriers spontaneously move in a direction opposite to gravity in the liquid in which they are suspended. In one of many possible embodiments, the manufactured microcarriers are doped with both paramagnetic particles and glass bubbles exhibiting net positive buoyancy. These substances impart physical properties to the microcarriers previously unknown in cell culture. Furthermore, the density of the microcarriers may be controlled by using various combinations of ingredients, some with buoyant properties, such that that the density of the carrier for cell culture is within the range of 0.8 to 1.4 g/cm, which allows suspension of the microcarriers in a culture medium.

### Manufacturing

Microcarriers can be manufactured using a plurality of methods, including but not limited to spraying, sonicating, suspending, vibrating, or emulsifying the liquid containing the raw materials from which the microcarriers are polymerized, in suspension, and in oil water emulsions. Imparting the engineered properties described in this patent, such as the ability to control buoyancy, is accomplished by adding selected material to the microcarrier raw materials, such as glass bubbles, so that they distribute themselves in the microcarrier according to the needs of the user. Alternatively, the material that imparts selected properties to the microcarrier may be added after the microcarrier has been manufactured.

Special proteins can be incorporated into the matrix of the microcarrier or in the surface coating of the microcarrier that will promote or enhance cell adhesion, growth, differentiation, or promote expression of a selected phenotype including morphological changes as well as the expression of biochemicals. For example, (but not limited to) microcarriers into which extracellular matrix proteins have been incorporated, such as collagens, fibronectins, peptides, and other proteins and biochemicals that may have been used to induce a variety of cellular behaviors (including those mentioned above). Alternatively, non-specific adhesion and cellular behaviors have been inhibited through the use of polymers, biochemicals, and other substances (10-14). Gelatin has been used to promote cell adhesion to planar glass slides (15). Prior art teaches a low density collagen coated microcarrier method for culturing, harvesting, and using anchorage dependent cells (16). However, the present invention discloses the creation of microcarriers that can be automatically manipulated by non-impeller based methods with engineered buoyancy to match the needs of the cells to be cultured. Furthermore, the engineered microcarriers of the present invention can be used directly in applications that call for living cells, which is different from what is taught by Hillegas (16), who describes insoluble microcarriers that are not optically clear. The Hillegas proposal does not teach the use of any specific cell or cells and the inherent advantages of their invention for supporting growth of particular cells. The present invention improves upon the teaching of Koichi (10) that allows non-adherent cells to attach to glass slides for microarrays. The engineered microcarriers of the present invention improves upon the method of Koichi in that they are suspendable, engineered with additives, and participate in a cell culture process which takes advantage of their ability to be manipulated in suspension. The advantage of these anchors is that they allow a plurality of non-adherent cells, such as blood cells, immunocytes (cells of the lymphoid series which can react with antigen to produce antibody or to become active in cell-mediated immunity or delayed hypersensitivity reactions; also referred to a immunologically competent cells), some cancer cells, stem cells, single cell organisms, and other cells to anchor to a variety of substrates (10). In the case of the present engineered microcarriers, in some embodiments, the biocompatible anchor material is incorporated in the matrix of the microcarrier, or on a surface layer coated onto the microcarrier according to procedures described herein. In one embodiment, an anchor promoting material is oleyl poly(ethylene glycol) ether (10). Including this anchor promoting material with the engineered microcarrier that can be manipulated in suspension, one has a powerful cell culture technique that will work with virtually all anchorage dependent and independent cells.

Alternatively, cells can be held inside an engineered microcarrier in a microenvironment that allows for cell differentiation or growth, or maintains the cell in a steady state non-growth phase. The engineered microcarriers could then be used to deliver the cell to a selected location, such as transplantation in a living being, where the cells would then be allowed to attach, differentiate into other clonal cell lines, or expand to fill a space or need. A breakable or enzymatically digestible biocompatible microcarrier may be used allowing the cells to be delivered to a site of interest and then the bubble would be digested, broken, collapsed or dissolved by a variety of means. An example of this latter method, one could break the bubbles by delivering ultrasound energy to the same location as the bubbles either in-vitro or in-vivo.

More specifically, microcarriers may be manufactured from a number of substances including two major classes of material, namely thermoplastic polymers, hydrogel polymers. Thermoplastics are any water soluble substances including but not limited to polyacrylates or polyethylene glycol. It is advantageous to use more gentle, and thus less harmful, manufacturing conditions for cells that are encapsulated in hydrogel substances, such as, but not limited to agarose and/or alginate. As a specific but not limiting example, alginate is an intracellular matrix polysaccharide extracted from brown algae and some bacteria. In order to improve the viability of cells on our unique engineered microcarriers we selected alginate from sources that do not contain endotoxins. Even those skilled in the art of cell culture on alginate microcarriers would benefit from our teaching the use of low or no endotoxin containing alginate in order to improve cell attachment, health, growth, and viability on the microcarriers. Alginate is obtained from either Sigma (St. Louis, Mo.) or Pronova Biomedical (Oslo, Norway) and mixed in an aqueous solution using endotoxin free water in a range from 0.1% by weight sodium alginate to 10% sodium alginate. However, the microcarriers are easier to manufacture due to the viscosity of the solution when the alginate concentration was 0.8% to 1.2%. Endotoxin is measured using a Limulus-lysate assay kit from Sigma and only alginate solutions with values of less than 5000 endotoxin units/mL were used for manufacturing microcarriers with cells on their exterior. The ideal range for culturing cells was an endotoxin level of less than 500 endotoxin units/mL. The alginate solution is mixed with a 2-4% by weight propylene glycol alginate (PGA) solution (Kelcoloid™ D; ISP Alginate, San Diego, Calif.) in order to crosslink the alginate [Kwon et al. (7)]. This optional procedure can be performed with concentrations of PGA from 0% to 10% by weight. In addition, alginate is created by its living source with regions of mannuronic acid or guluronic acid, or a mixture of both mannuronic and guluronic acids. Sources with ratios of these substances that optimize cell health, growth, and viability are selected. Ratios of mannuronic acid to guluronic acid are determined by emission at 445 nm according to the method of Klöck (17). The preferred ratios for crosslinking with calcium is 90% or greater mannuronic acid although cell growth is observed at any ratio of mannuronic acid to guluronic acid. Additional additives include glass bubbles (3M Corporation, Maplewood, Minn.), protein bubbles, or air bubbles up to 20% by volume. However, we find that glass bubble 1%-5% allows for microcarriers with ideal densities and that are affordably manufactured. Air (or other inert gas such as helium) bubbles can be incorporated into the solution by vigorously shaking the container to trap air in the form of small non-uniform bubbles or by using bubbler (compressed air or gas is pumped into a sintered metal device that divides the gas into uniform bubbles). Paramagnetic or ferromagnetic particles (Spherotech, Libertyville, Ill.) can also be included in the solution at this point in order to impart paramagnetic or ferromagnetic properties to the resulting microcarriers. Up to 75% of the internal volume of the microcarrier can be filled with para or ferromagnetic particles, however, the idea ratio is from one to 1000 particles per 250 um microcarrier. Indicators, such as fluorescent molecules described elsewhere in this application can be added, if desired, at this point.

Once the contents of the microcarrier solution has been determined, based on the specific properties of the resulting microcarrier desired, the microcarriers are formed by a variety of methods including adding the alginate/PGA additive solution drop wise to a gently agitated 1.5% (0.135M) calcium chloride solution. Commercial micro droplet generators may also be used. Living cells may be added to the mixture before the microcarriers are created to allow interior cell encapsulated culture or the co-culture of cells both inside and outside of the microcarrier using either similar or dissimilar cells. The alginate solution is adjusted with a physiologic buffer if living cells are intended to be encapsulated. Microcarriers are then used for cell culture after washing. Microcarriers can also be coated with gelatin by adding a 1% gelatin solution (for example unflavored Knox gelatin from the local grocery store) to any volume of a microcarrier suspension, gently mixing, and then washing the beads by repeated changes of fresh buffer. High concentrations of gelatin add greater rigidity, thus we have used up to 10% gelatin, but 0.5% to 3% is ideal for cell attachment. Additives are placed in the gelatin solution including molecules that enhance cell attachment, molecules that can transform cells (DNA, RNA), and indicators as described elsewhere in this application. The Gelatin can be crosslinked to give microcarriers with greater rigidity by transacylating to the alginate by adding two volumes of 0.2M NaOH as described by Kwon et al. (7). Various molecules are incorporated to increase or decrease the microcarrier charge and/or porosity, such as but not limited to poly-L-lysine (a cationic amino acid polymer) (18). The present invention discloses the incorporation of substances that control microcarrier response to physical forces, which improves upon the use of substances that control microcarrier permeability, porosity, and strength.

The alginate guluronic molecules and hence the microcarriers are held together and develop rigidity and hence strength through the addition of bivalent cations such as calcium (Ca²⁺). Both the guluronic and mannuronic acids are bound together using Barium (Ba²⁺), so the incorporation of Ba²⁺ is important to achieve stronger microcarrier properties according to Strand. (19). As apposed to the art taught by Strand to use selected cations to increase microcarrier rigidity, we teach the novel art of using Ba²⁺ as a bivalent molecular bond in order to avoid the use of Ca²⁺ in biochemical assays examining calcium flux and concentrations because changing calcium concentrations would both disturb the measurement and would alter the integrity of the microcarrier.

Microcarriers are manufactured using a variety of methods including the use of an electromagnetic or piezoelectric driven nozzle equipped to allow laminar-jet-breakup of the alginate solution and additive suspension. The use of a commercially available encapsulation system is desirable to allow control of the physical parameters affecting microcarrier size (e.g. flow rate, vibration frequency and amplitude). Alternatively, the alginate solution is added to a rapidly stirring emulsion of oil and buffer containing bivalent cat-ions or cross linking agents. The microcarriers spontaneously form their micro-spherical shape in the emulsion and then precipitate out of the oil when the stirring is slowed or stopped if they have a density greater than that of the emulsion buffer. If the microcarriers are made with a buoyant density, then they can be removed from the emulsion by centrifugation and aspiration from the surface, or by pulling them to the side of the vessel if they contain paramagnetic particles.

### Using Engineered Properties to Facilitate Using Microcarriers

Growing cells on standard microcarriers that are suspended in the growth medium allows greater access to nutrients, air, or oxygen, and carbon dioxide and random orientation with respect to gravity, yet increases potential damage due to uncontrollable shear stress. An added benefit of the engineered microcarriers of the present invention is that they provide gentle growth conditions without the stresses or inconveniences imparted by stirred cultures. The present engineered microcarriers allow specific orientation of each microcarrier to be externally controlled. Additionally, the engineered microcarrier can be easily and quickly harvested for subsequent procedures. The amount of microcarriers that may be used to grow cells is limited only by the amount of culture media in the vessel. Thus, the use of the disclosed engineered microcarrier allows for culture scalability from use in a microscale culture in microfabricated technologies (20) to culture systems in excess of one liter, for example 500 Liters.

Engineered microcarriers have been successfully created that were approximately 5 um which became partially engulfed by one Chinese Hamster Ovary cell during its growth. This technique will allow cells to remain on their anchorage surface while being translocated in a microchannel fluid stream or immobilized either temporarily or permanently on a flat surface, three-dimensional surface, or array.

Furthermore, the present invention discloses the use of microscale components, such as micromagnets, micro-pressure systems, and micro-detectors to perform many of the same procedures described in this patent application, only on the microscale. An important advantage of the present invention is the ability to steer cells within microchannel arrays using pressure or magnetism, to which the engineered microcarriers will respond. The present invention comprising a suspension culture of engineered microcarriers increases the productivity one can expect, of conservatively 400 fold over flat bottom dishes, and over two fold compared to spinner flasks.

Once cells have reached a desired level of confluence, for example 80% coverage of the microcarrier, it is often necessary to remove the cells from the microcarriers in order to use them for analysis. The most popular method of removing viable cells from their anchor surface is through the use of a proteolytic enzyme (trypsin) that digests some of the proteins used by the cell to anchor them on the microcarrier. Not only does trypsinization strip the cell of many important cell surface proteins and it causes a temporary shock to the cells, often resulting in a low yield of cells that are released intact from the microcarrier. Also cells may need a mechanical shock (such as the energy imparted by rapid deceleration of microcarriers in solution) in order to be released from the microcarrier. The more dimpled the surface of the microcarrier, the less likely those cells will release from the surface or be sheared off. The specific art of releasing cells from microcarriers was addressed by Mundt (21), who taught the use of trypsin to release the cells from the microcarriers. The present invention does not have these problems as the present microcarriers are engineered to dissolve spontaneously, as described by Kwon (7), thus obviating the challenges associated with using non-specific enzymes to release cells from their anchorage surface. Thus, the present invention is intended to encompass the use of spontaneously dissolving engineered microcarriers that work in concert with automation to obviate the need to perform these tasks manually. Furthermore, the ability to dissolve the microcarriers within a specific time point and location within an automated process has not previously been described. For example, engineered microcarriers can be dissociated either partially or fully during their transition in a fluid stream prior to analysis in a cell sorter or fluorescence activated cell scanner. Our engineered microcarriers may be more quickly dissociated by making use of the external control of the properties of the microcarrier. For example (but not limited to), increased internal kinetic energy imparted by rapidly moving magnetic or paramagnetic particles as the result of an externally applied oscillating magnetic field can quickly dissociate the polymerized alginate when calcium is reduced below the polymerization threshold in solution. By dissolving microcarriers containing paramagnetic particles and/or glass bubbles, one can retrieve these substances for either reuse, or to prevent them from contaminating or adversely affecting downstream processes.

Another benefit of the engineered microcarriers is that they may be used in conventional cell culture facilities employing conventional disposable dishes, pipettes, culture media, incubators, cell dispensing equipment, rockers, agitators, plate sealers, and analytical instruments.

### Imparting Movement in the Growth Media

Agitation of microcarriers has been traditionally performed through the use of impellers (see above). But there are many benefits of stirring growth media without the use of impellers. The present invention provides alternative approaches to imparting kinetic energy to growth medium containing living cells grown through the use of the engineered microcarriers. Imparting kinetic energy to the growth medium assures even distribution of nutrients, assures good gas exchange to all cells, and prevents clumping of engineered microcarriers. The present invention provides a number of methods to impart kinetic energy to the growth media that may be used individually or in any combination. For example, the kinetic energy may be in the form of using a heat source that induces a thermal gradient in the growth medium. The thermal gradient imparts motion in the growth medium as less dense heated media rises in the culture vessel, the more dense cooler media tends to sink in the culture vessel and hence imparts movement of the engineered microcarriers. The thermal gradient is sufficient to induce kinetic energy, but not cause harm to the growing cells which generally can tolerate 33° F. to 105° F. unless they are cryoprotected or thermally stabilized, respectively. A temperature differential from ambient of one degree to greater than 40° F. above ambient may be used to induce convection currents. The thermal gradient can be controlled by a servo controller so that it actually serves as the heat source to warm the culture medium to temperatures that impart optimal cell growth.

Pressure can also be applied to the microcarriers can achieve two goals. The first goal is to subject the cells growing in or on the microcarrier to a pressure profile similar to that felt by cells growing in living beings. Thus pressure pulses, or differential pressures over time, can be applied to the microcarriers at rates found in nature such as 5 beats per minute up to 500 beats per minute. The second goal of the applied pressure force is to compress gas bubbles incorporated into the microcarrier to enhance buoyancy. By compressing the entire container in which the gas bubble containing microcarriers are held, one can increase the density of said microcarriers thus causing them to sink under applied pressure, and rise under reduced pressure. Pressure from ambient barometric pressure up to many atmospheres may be used.

### Mechanical Modulating Microcarrier Buoyancy

Another embodiment of the present invention exploits particle buoyancy to increase the kinetic energy in the culture vessel or bioreactor. For example, particles are introduced that are either composed of compressible gas bubbles, or contain compressible gas bubbles. A variety of natural or man-made elastic materials may be used to trap a gas bubble(s). Since gas is more compressible than liquids, compression of the gas by the use of an externally generated energy source, either thermal or pressure will impart varying buoyancy to the particles. Particles exhibiting variable buoyancy may be exploited to either stir the growth medium containing microcarriers supporting cell growth or maintenance, or compressible bubbles may be introduced into or on the microcarriers containing cells.

### Modulating an External Magnetic Field

Magnetic fields may be used to induce kinetic energy into a fluid, such as cell culture medium. A large magnetic flux induces movement at the microscopic, and ultimately, at the macroscopic level in any liquid. Alternatively, in order to limit the amount of magnetic field that has to be generated, in one embodiment, the present invention discloses the introduction of ferromagnetic or paramagnetic particles into (or on) the microcarrier whose motion can be induced by an externally generated magnetic field. Ferromagnetic particles constitutively exhibit a magnetic field, whereas paramagnetic particles only exhibit a magnetic field while being exposed to a magnetic field. The motion of the particles induces motion in the liquid, and hence maintains the suspension of microcarriers supporting the growth of cells. The paramagnetic particles may be attached to the surface or placed inside the microcarrier that are supporting the growth or maintenance of cells which are grown either inside or on the surface of the microcarrier to produce an example of an engineered microcarrier within the meaning of the present invention. Ferromagnetic material, or any material that responds to a magnetic field, can be placed in or on the microcarrier by adding formed particles or precipitating the material from solution during the microcarrier manufacturing process, or introducing it as a coating once the microcarrier is manufactured. Known magnetic materials include, but are not limited to chromium, iron, nickel, and cobalt and their oxides and derivatives. These materials can be added from 0-75% by weight in finely divided nanoparticles so as to provide less interference with optical properties, or as a large core so that the optical properties of the perimeter of the microcarrier is preserved.

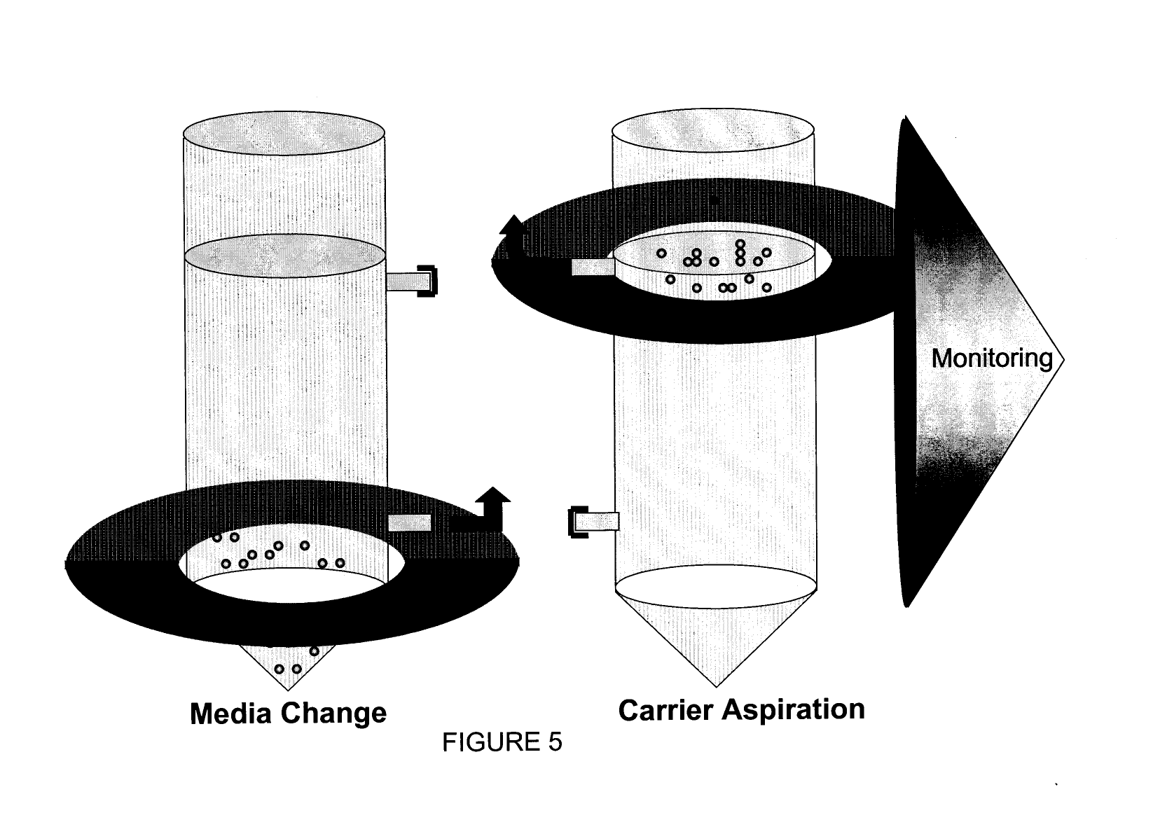

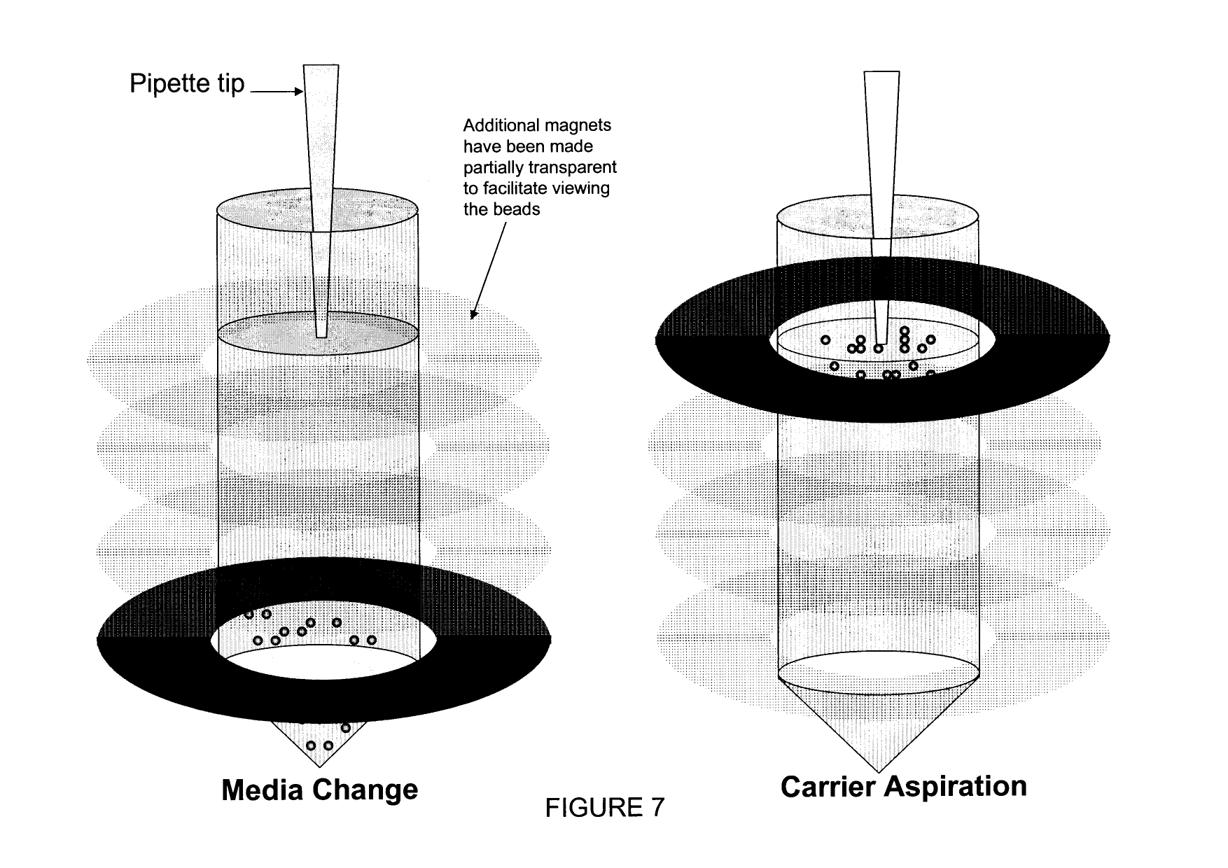

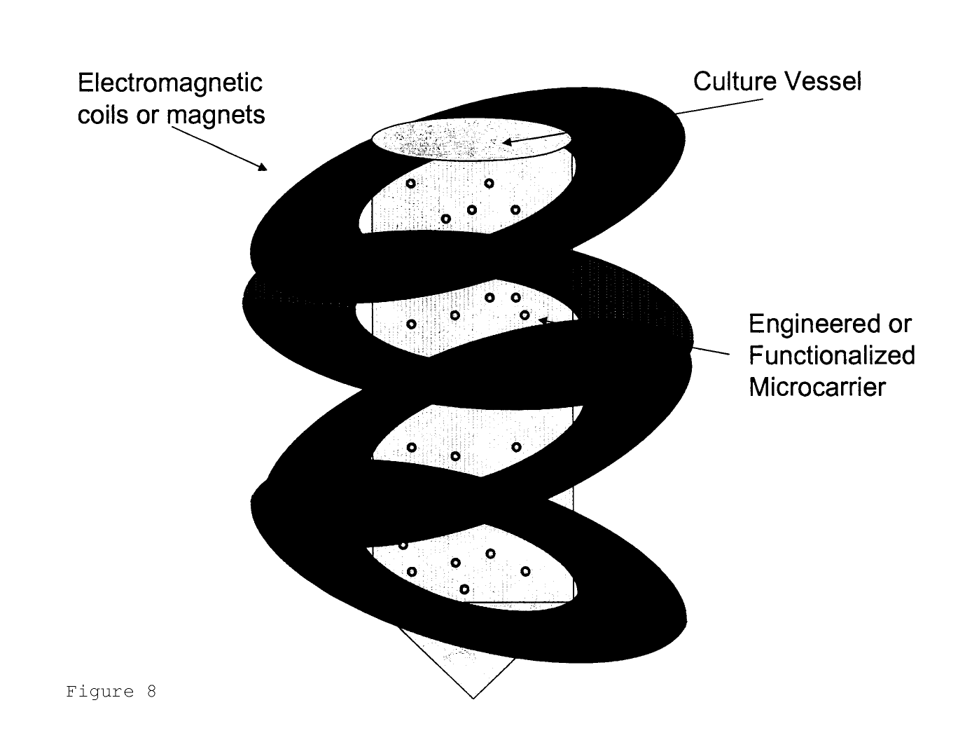

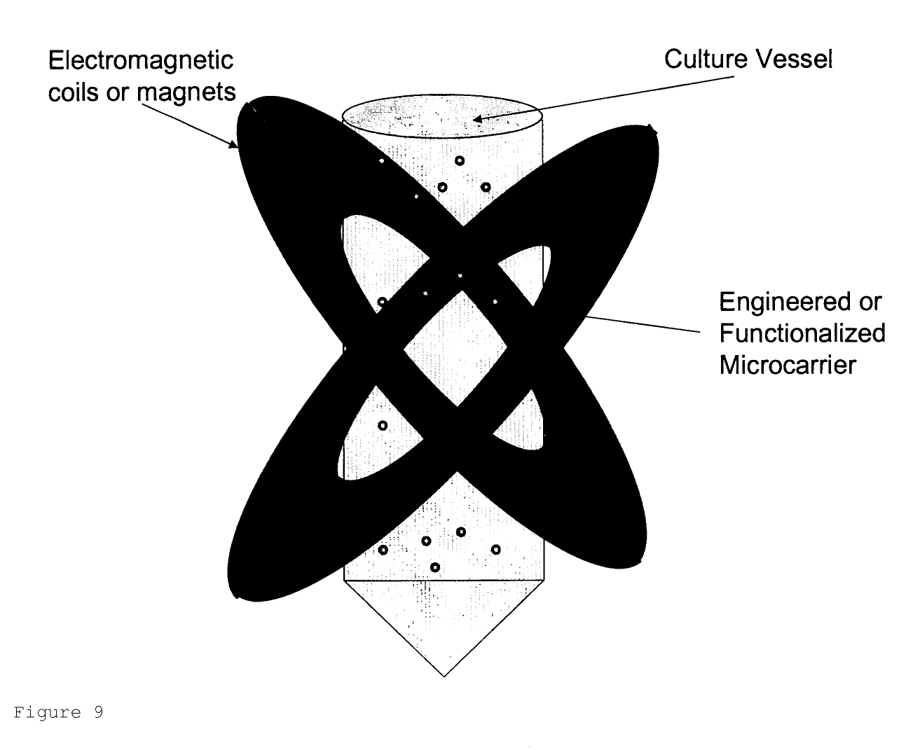

The magnetic field may be modulated by either use of permanent magnets, or electromagnets placed above, below, or on the side of the cell culture vessel. The placement of the magnet will depend on the movement desired from the microcarriers. The magnetic fields may be continuously applied when a specific microcarrier orientation within the vessel is desired, such as (but not limited to) bringing the engineered microcarriers to the bottom of the vessel to allow the media to be aspirated, or bringing the microcarriers to the surface of the media so that they may be harvested. (See FIGS. 5 and 7) Magnetic fields may be applied with different temporal or strength profiles. For example (but not limited to), pulsing the magnetic field is useful for maintaining the microcarriers in suspension (See FIGS. 5-10), yet limit the amount of heat generated by an electromagnet, or the amount of mechanical movement of a permanent magnet. Hybrid magnetic fields may be applied, such that a field with deep penetrating strength may impart selected movement or orientation of the microcarrier, while at the same time a stronger field with less penetrating strength may be used to hold microcarriers in a selected orientation.

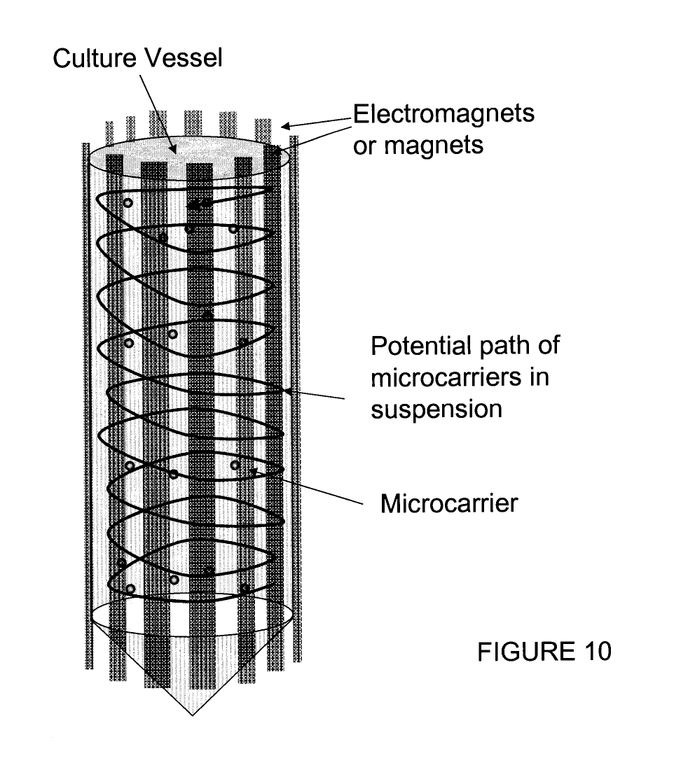

FIG. 10 shows one embodiment in which the verticle bars represent bar magnets arranged in a circular fashion around the perimeter of the vessel. By computer-controlled activation of the magnets and alteration of their polarity, many different microcarrier paths can be effected for stirring, media changing, cell adhesion operations, or cell harvesting.

A combination of the above techniques can be used to optimize the growth or maintenance of cells, depending on the cell type and growth conditions required. For example, in one embodiment, the present invention utilized both paramagnetic particles and bubbles introduced into the same microcarrier simultaneously to obtain an engineered microcarrier with a blend of properties that both the paramagnetic particles and bubbles impart to the microcarrier. This combination of paramagnetic particles and bubbles imparts the ability to control buoyancy as well as the ability to use a magnetic field to stir and direct the movement and/or orientation of the magnetic particles in the bioreactor. Thus, engineered microcarriers may be manufactured to match the specific needs of each cell type, depending on the needs to control kinetic energy, density, response to the externally applied magnetic field, and orientation. For example, an external magnetic field could be applied to the culture media containing cells and the engineered microcarriers so that an engineered microcarrier with buoyant properties would be attracted to the bottom of the culture vessel to allow initial cell attachment. The magnetic field could be then removed to allow buoyant engineered microcarriers with growing cells attached to rise into the growth media.

### Use of Populated Engineered Microcarriers Directly in a High Throughput System (HTS)

Once engineered microcarriers of the present invention have been populated with cells, they may be used directly in biochemical or physiologic procedures. The ability to maneuver the engineered microcarriers to a specific location as a result of their inherent properties or through the use of automation allows them to be exploited for use in subsequent research or development procedures. For example, the use of their buoyancy and/or heat convection to cause the engineered microcarriers to move to the upper portion of the cell culture vessel or bioreactor will make them available to an automated pipette, or other means of harvesting cells. Alternatively, the engineered microcarriers can be gathered at the top of the bioreactor by an externally applied magnetic field or induction of lower density for aspiration by a pipette. Another embodiment of the invention is to maneuver the microcarriers to a liquid port in the bioreactor so that they are concentrated and pumped out in the fluid stream to be used in a subsequent procedure.

### Functionalized Microcarriers

Biochemical procedures or analyses on cultured cells grown in cell culture laboratories have a variety of uses, including research, product development, and drug discovery. Normally, cells must be digested or otherwise dissociated and fractionated so that one can study individual organelles of the cells, or biomolecules produced by the cells. Recently, “high content” discovery or screening has resulted from the ability to study whole cells. Novel cell culture plates have been developed to allow cells adherent to a surface to be examined by intracellular fluorescent reporting molecules. However, cells grown on flat dishes do not have similar phenotypes or behaviors as compared to cells in situ. In contrast, cells grown and maintained on microcarriers have been shown to be polarized, demonstrate phenotypes more equivalent to their in situ counterparts, and produce larger quantities of cell products. Cell sorters or fluorescence activated cell sorting (FACS) instruments have been developed to study cells in suspension. Suspended cells are moved in a narrow stream of fluid in front of an optically based detector in order to quantitate size, fluorescence, and/or electrical properties. Unfortunately, when anchorage dependent cells are placed in an environment where they are not anchored, they often exhibit negative properties. The presently manufactured engineered microcarriers are small enough so that individual cells are supported by the microcarrier matrix. Thus, the engineered microcarriers of the present invention are useful for cell counting and sorting instrumentation. The paramagnetic and buoyant properties of the engineered microcarriers are also useful as a means to separate cells from their liquid environment, to sort cells, or to measure responses to stimuli.

An enhancement to conventional non-engineered microcarriers or to the engineered microcarriers described herein is to functionalize the microcarriers so that they contain ligands and or binding molecules that report a stimulus and/or respond to a stimulus. For example, microcarriers containing a contractile protein may be induced to contract and change its buoyancy in response to a stimulus from the cell, or the cell culture bioreactor controller. Ligands, reporters, or response elements may be covalently or non-covalently linked to the surface and/or interior of the microcarrier. Reporters may consist of micro (or nano) electronic or micro (or nano) mechanical elements that are incorporated in or on the microcarrier which report via electromagnetic methods, for example but not limited to wireless motes. Ligands may be used to cause a reaction from the cells grown on any aspect (outside, inside, or both) of the microcarriers. Microcarriers may be functionalized with reporters so that they report changes to their environment as a result of changes in the culture media or changes resulting from materials exported or secreted from the cells. In one embodiment, reporters can signal the presence of and progress of a reaction, or a response to a stimulus. A large number of light emitting reporters are available in the form of fluorescent and bioluminescent molecules. The choice of reporters will vary according to what is to be measured. For example, in one embodiment, a sodium sensitive reporter for sodium can be placed inside the cell to report intracellular sodium. Similarly, a sodium sensitive dye can be incorporated into the microcarrier so that sodium pumped by the cell to its anchorage surface on the surface of or into the microcarrier would be reported. The reporter may be organic, inorganic, and single or multiple molecules, linked directly to/in the microcarrier, or linked to a functional group which was first linked to/in the microcarrier. Our process of functionalizing microcarriers to report or respond differs from the prior art described (15) in that our microcarriers are designed to support living cells which release molecules of interest. Furthermore, the present invention discloses the use of molecules that respond and alter the microcarrier environment, such as in one embodiment, contractile elements.

Engineered microcarriers may be used for a plurality of assays that are of interest to pharmaceutical companies and basic researchers. For example, there is great interest in determining the ability of cancer cells to metastasize, and to determine the mechanisms cells use to bind, penetrate, and move into foreign tissue. Cell migration and/or metastasis assays are useful to find or refine new anticancer agents, or examine how arteries form in developing tissues. The engineered microcarriers disclosed herein are designed to measure cell migration or invasion based on biochemical assays. In one embodiment, cancer cell division into the microcarrier may be monitored by measuring cell number or a signal emitted as a result of cell division. For example, in this embodiment, reporter molecules sensitive to cell surface proteins can be polymerized into the core of the microcarrier. As cells, growing on the surface of the microcarrier penetrate toward the core an increase or decrease in the fluorescence signaling molecule is measured. Thus, signal magnitude is correlated with the ability and avidity of cells to migrate or invade. In another embodiment, the microcarrier is coated with a substance that resembles basement membrane or other biological barriers that may be invaded by cells growing on the microcarrier. Cells are co-cultured on the surface and/or interior of the microcarrier so that invasion is measured from an outer layer of cells toward the center of the microcarrier, or cells are observed and measured invading outward, away from the core of the microcarrier. Alternatively, the microcarrier containing the potentially invading or migrating cells are attracted toward other cells growing on another microcarrier (using a magnetic field or buoyancy) to observe and measure invasion or migration from one microcarrier to another. Microcarriers additionally may be attracted toward cells growing on a conventional anchorage dependent surface, for example, in a further embodiment, the surface of a conventional culture flask, using gravity, buoyancy, thermal gradients and/or magnetism. Once they have come within a specified distance, then cell migration or invasion from the surface to the microcarrier or from the microcarrier to the surface is measured.

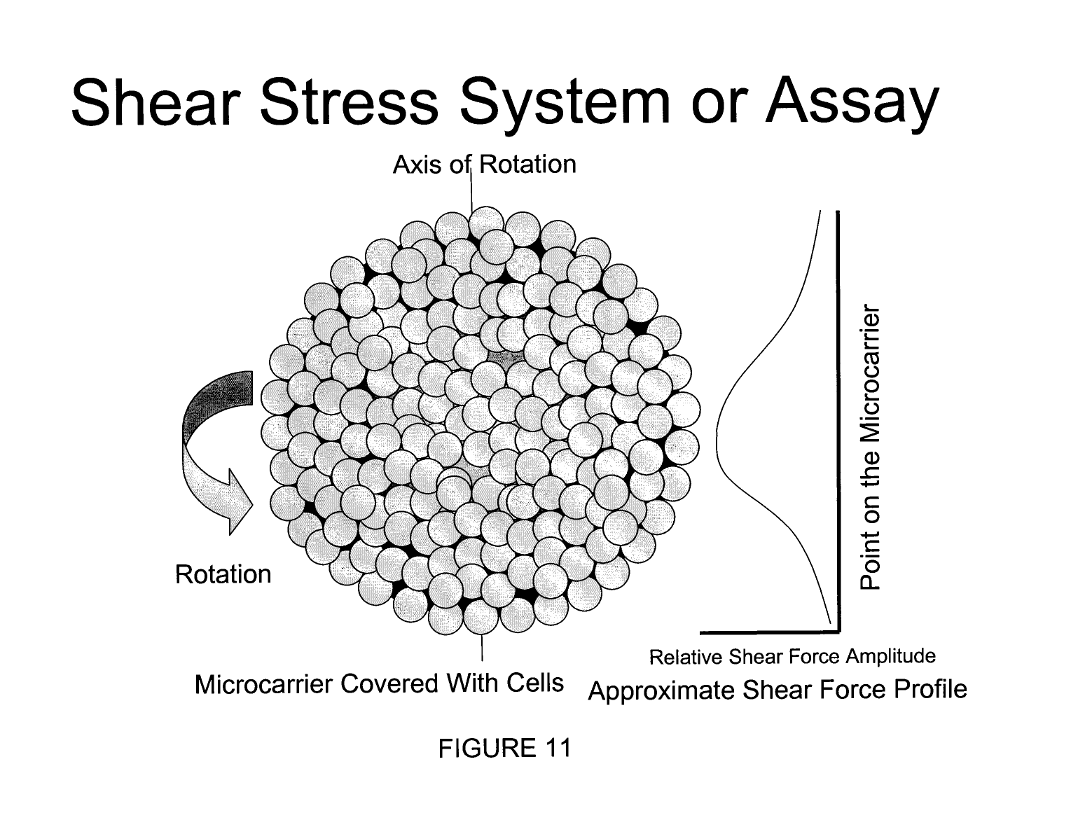

The effects of shear stress on cellular physiology or biochemistry are measured using the engineered microcarriers of the present invention. A rotating microcarrier will impart shear stresses on the cells on its surface (See FIG. 11). Thus, changes in cellular physiology or biochemistry are measurable in response to an externally applied magnetic field that allows for changes in microcarrier internal or external kinetic energy, for example, in one embodiment, rotation according to a user programmable profile of speed, direction, amplitude, and temporal profile (such as pulsatile, ramping, square wave, and other user definable profiles).

Engineered microcarriers of the present invention are useful to mimic the blood brain barrier. The brain is a difficult place to deliver active pharmacological compounds. The blood-brain barrier has been actively studied to determine how this barrier separates the brain from the circulating blood. Thus, the engineered microcarriers and the culture system of the present invention provides a model for pharmaceutical discovery in methods that can mimic the blood brain barrier and allow its study, as well as the development of an in-vitro model of the blood brain barrier. In this embodiment, brain vessel endothelial cells are grown on engineered microcarriers that are useful to determine how much of a selected compound within the culture media gains access to the interior of the cells and/or microcarrier core or compounds inside the microcarrier gain access to the interior of the cells or get exported to the exterior of the microcarrier cell layer to an reporter layer or the media. This model can be easily deployed in any laboratory using engineered microcarriers.

In a further embodiments, engineered microcarriers are used as an attachment surface for stem cells that are derived from a variety of sources such as (but not limited to) cord blood, adipose tissue, embryos, and peripheral circulation. The simulated microgravity environment is favorable for promoting the maintenance or differentiation of stem cells into progeny cells.

### Bioreactor

More than 100 biopharmaceutical products are currently approved for use in humans by the FDA, creating a market of over $100 billion, with an annual growth rate of over 100%. Bioreactors or culture vessels are used to produce proteins under conditions that are optimized for cell growth (22-31). Once cells have reached maximum density in a bioreactor, competition for nutrients and oxygen causes cell death, which leads to system inefficiency. Most bioengineers consider the bioreactor as having reached maturity, and thus are seeking more efficient and optimal processes. Hollow fiber bioreactors (or perfusion based systems) have improved protein production, but only for cells that secrete the protein of interest. Hollow fiber systems become clogged with the products of dead cells as the culture matures, leading to lower yields compared to many batch systems. Thus, until now, no one technique has yielded optimal cell viability and protein productivity.

Bioreactors are operated for as long as 120 days in order to produce proteins of interest. Therefore, there is a significant amount of labor in monitoring and maintaining optimal reactor conditions (pH, nutrient level, temperature, dissolved gas concentrations). Generally, cells are not removed from the bioreactor. These large batches are maintained by adding nutrients or adjusting conditions as the process continues. There are resulting monitoring gaps as liquid is removed from the bioreactor and sent to the laboratory for analysis. Ideally, monitoring of cell growth and metabolism should occur in real time, at the cellular level.

The automated cell culture system of the present invention comprises engineered microcarriers as described herein which have an indicator imparted into their structure that would allow each engineered microcarrier to report the health and growth conditions for the cells growing on its surface (or interior). Through the use of indicators, a closed loop control system would be able to be implemented on each of the bioreactor modules. In our embodiment, the engineered microcarriers of the present invention are engineered to report microscopic conditions at the cellular level by incorporating indicators into the matrix of the microcarrier itself. For example, such indicators my be but not limited to, fluorescent indicators for pH, and indicators for oxygen, carbon dioxide, glucose, urea, bicarbonate, lactate, and ammonia can be incorporated into each microcarrier and monitored through the bioreactor or culture vessel. Alternatively, a conventional flow through analytical system can be used to monitor the components of the culture media.

The bioreactor of the present invention capitalizes on the ability of the engineered microcarriers to be agitated, rotated, heated, cooled, gassed (with unique gas mixtures), pressurized, exposed to magnetic fields (either constant or varying in any portion of the electromagnetic spectrum including, but not limited to the near infrared to far ultraviolet), in order to move and stir microcarriers.

Another embodiment of the present invention is an engineered microcarrier based bioreactor that comprises a single or a plurality of orifices or openings that maintain disposable cell culture vessels upright (or vertical) or laying on its side (or horizontal). The microcarriers may be introduced into the cell culture media contained in the bioreactor through one of the orifices or openings in order to affect an increase in kinetic energy within suspension cell cultures of non-adherent cells, such as for example, SF9 insect cells, which is derived from Spodoptera frugiperda. The bioreactor also comprises at least one source for generating at least one physical force to which said microcarrier is responsive.

In a further embodiment, the bioreactor described above contains an further element or elements necessary to levitate and manipulate the microcarriers in the growth medium, termed a control system. The control system may consist of hardware that is operated manually. The control system may be enhanced to include mechanical systems that operate automatically. The control system may be further enhanced to include software, and control electronics to enable a fully automated system to operate. For example, hardware, and control electronics and software would provide the heat elements whereby the microcarriers would be levitated by the thermal gradients (a thermal control system). The bioreactor would contain the hardware, control electronics and software to provide magnetic fields whereby the microcarriers would be moved, rotated, and/or held stationary (a magnetic control system). Magnetic fields could be varied by moving permanent magnets using robotic devices, servos, and other means. Alternatively, fixed or movable electromagnets under software control could be employed to manipulate the microcarriers. Hardware, and control electronics and software would provide the means by which pressure transducers could alter the pressure on the bioreactor to impart changes in microcarrier density through compression of gases contained in or on the microcarriers (a pressure control system). Either temporal or special pressure gradients or profiles can be imparted on the vessel to mimic biological shear or compressive stresses to study cell responses, or to induce cells to produce specific proteins or exhibit selected behaviors. Each of these control systems can operate on a single bioreactor, or a single control system could impart its action on a plurality of bioreactors. Alternatively, a plurality of control systems can operate on a plurality of automated bioreactors. The capacity of the bioreactor can be increased by simply increasing the size or number of bioreactors.

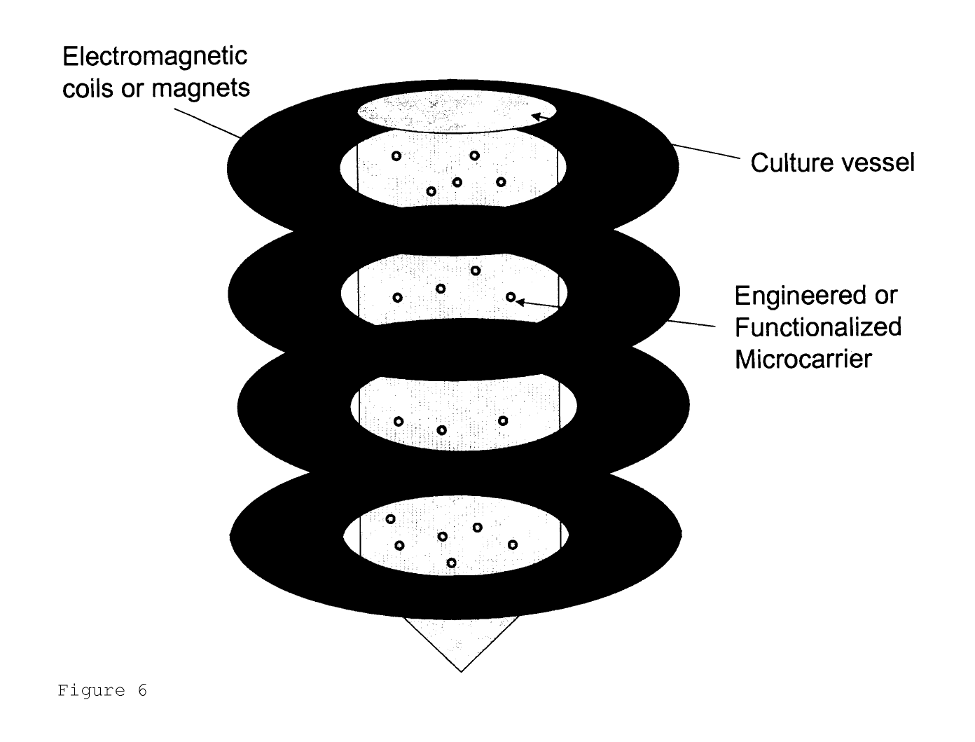

The use of magnetic fields to manipulate microcarrier orientation and/or movement is different than the use of electromagnetic fields to stimulate the attachment of cells to microcarriers as taught by Wolf (32). In the former case our magnetic fields impart changes in the kinetic energy of the microcarrier, in the latter case Wolf is enhancing the attachment of cells to microcarriers. In one embodiment, the present invention uses individual, linearly spaced electromagnetic coils oriented at right angles to the bioreactor to generate a linear magnetic field suitable for maintaining ferromagnetic microcarriers in suspension (See FIG. 6). The magnetic flux and shape of the lines of magnetic force can be varied by controlling the current, radius of the coil, the number of windings in the coils, diameter of the wire, number of coils, and the spacing of the coils. In order to use an electromagnetic approach, the current can be varied between 0.1 amps and 100 amps. Windings can be varied from one to as many windings that will fit in the space surrounding the cell culture fluid column. The spacing can vary so that only one coil or hundreds of coils are in a 15 cm length. The diameter of the coils can be as small as the diameter of the cell culture tube to as wide as possible so that a magnetic flux can still impart movement to the microcarriers. The use of magnetic coils to control paramagnetic microcarriers has been previously taught (33). However, they teach the use of this technique to hold microcarriers containing enzymes stationary in a moving field so that waste water may be purified, not cells cultured.

### Automation

The cell culture process is a tedious and labor-intensive undertaking that has a high error rate and is prone to contamination by the people managing the process. Many cell cultures and cell culture facilities are contaminated with mycoplasma, fungus, yeast and other organisms usually derived from the individuals performing cell culture. Cell culture involves countless hours spent by researchers and technicians in a sterile environment feeding and sub-culturing living cells. In addition to the labor costs, cell culture is an expensive process consuming large quantities of sterile plastic pipettes, culture dishes, media bottles, and other associated materials.

Robots have been used to automate (34-36) the steps currently performed manually (37). For example, in performing conventional cell culture, cells are first thawed from frozen stocks that are maintained from −80° C. to −150° C. The thawed stocks are placed in cell culture media in a 12 mm by 75 mm sterile disposable culture flask (often called a T75). The flask is placed into an incubator to allow the cells to attach to the surface of the flask and to begin to divide and grow. Continuous feeding (e.g. three times per week) is necessary to maintain growth rates and cell viability. Feeding involves careful aspiration of spent media using a disposable sterile plastic pipette introduced into the culture flask. Fresh, warmed media is then carefully introduced so as to not disturb the growing cells. When the cells have reached the proper degree of confluence, then the cells can be removed from the flask for use. Removal of cells involves scraping or detaching by mechanical or enzymatic methods. In either case, cells are either physically damaged or denuded of cell surface proteins during these steps. In order to propagate the cells, the cells are usually enzymatically detached from the dish and frozen for long term storage.

The present invention discloses the automation of cell culture through the use of the combination of a microcarrier or an engineered and/or functionalized microcarrier for growing cells, a bioreactor that contains and supports the use of these microcarriers, and an automation system that provides for manipulation of microcarriers, fluids, gases, and bioreactor components. The automation system can also comprise a computer system equipped with process control software to manage the automated cell culture process and to provide data on the progress of the system. Pluralities of sensors are employed to monitor the actions of the automation system and the conditions of the environment so that feedback control of each process is maintained. The bioreactor requires the unique properties of the microcarrier, and the configuration of the automation depends on the properties of the bioreactor and microcarrier. Through the use of automation, many of the manual steps involved with cell culture of seeding, growing, feeding, splitting and assaying can be reduced or eliminated resulting in less contamination. In addition, since cell scraping or enzymatic digestion is not necessary using microcarriers, healthier cells may be introduced directly into a downstream process such as drug discovery. Continuous culture of cells for protein production is also supported by the automation system.

The automation system comprises microcarriers or a microcarrier making device, a bioreactor, an optional monitoring system, an optional control system, a method to move liquids, culture vessels, and disposable culture ware. The mechanical devices provide a means to gain access to permanent or disposable culture ware that supports the use of the microcarriers of the present invention. In one embodiment, at least one cup shaped plastic culture vessel that holds cell culture media and allows access by liquid handling equipment from above. The vessel may be maintained as an open container if the automation system is contained in a sterile environment. Sterility can be achieved in conventional ways including (but not limited to) the use of ultraviolet light to kill living microbes, pollens, and spores, airborne bacteria, fungus, and virus, or by using HEPA filters equipped to remove all particles over a specified size. Alternatively, the cell culture vessel may be closed, but equipped with an orifice or opening that allows entry and exit of a tool while maintaining closure, as in a septum. A septum is a device that is integrated into the culture vessel that acts as a port for adding or removing material from the culture vessel. The septum may be capped with a pierceable rubber cap that can be penetrated by a rigid pipette. When the pipette or syringe needle is removed, the rubber cap reseals. Culture vessels may be rigid allowing only gas exchange from the open end, or may be constructed out of a material that is engineered to allow free exchange of gases such as CO₂ and O₂. In one embodiment, polyfluorinated culture bags (American Fluoroseal Corporation, Gaithersburg, Md.) are utilized that have excellent gas exchange, but do not allow exchange or loss of liquid. The culture bags may be supported in any vessel that is designed to support a culture bag including a standard 50 mL centrifuge tube or a larger rigid structured container to hold the culture bag. In one embodiment, holes are drilled in a 50 mL centrifuge tube to allow free exchange of gases. The use of polyfluorinated bags allows the continuous manufacture and feeding of cell culture vessels within the automated system through the septum that is sealed into the polyfluorinated plastic bag and which is inserted through the cap of the tube or container in which the bag is held, and sealed to the cap. For example (but not limited to), a roll of polyfluorinated sheet goods could be formed into a culture vessel by laser melting (or welding).

The automation system comprises a means to move liquid in and out of the culture vessel. For example, an overhead Cartesian robot equipped with pipetting tools could be used to aspirate or replenish liquid from the culture vessel. Alternatively, a cylindrical robot, articulating arm, Stuart platform, or other robotic system may be equipped with liquid handling hardware. Further, in one embodiment, a means may be provided to use the paramagnetic properties of the engineered microcarriers in order to facilitate removal of the culture media. For example, the culture media can be removed after attracting paramagnetic microcarriers to the bottom of the culture vessel through the use of a magnetic force as previously shown in FIGS. 5 and 7. Once the media is removed, then the pipetting robot could replenish fresh media in the culture vessel by either using a pipette of sufficient volume, using multiple trips from the source of media to the culture vessel, or by using a pipette equipped with a pump to continuously dispense culture media into the culture vessel. The magnetic force may be removed from the engagement vicinity of the microcarriers before, during, or after the media replenishment activity. The automation system may contain all the necessary hardware to perform the culture operations, or may employ the use of a bioreactor (described above) to perform various steps of the culture process.

The culture vessel may also be equipped with an inlet and outlet port for liquids that are in direct connection with the culture media, either through tubing dipped into the opening of the vessel, or through direct connections to the culture vessel that are installed during the vessel's manufacturing process. Microcarriers may be moved away from the ports when liquids need to be pumped out or into the vessel, or they may be moved toward the port when it is necessary to collect the microcarriers. Movement of the microcarriers may be through convection, microcarrier buoyancy, or through the use of their paramagnetic properties.

The entire automation internal environment is maintained at the appropriate cell growth temperature, humidity, and gas concentrations suitable for each cell type. Alternatively, selected parts of the automation system may be environmentally controlled. The bioreactor system or subsystems may be used in the automation system to provide the appropriate conditions to optimize the use of the unique microcarriers.

The sequence of events that would transpire in an automated system would be similar to that experienced when performing manual cell culture. Initially, cell culture users would deliver a vial of frozen or growing cells to the automation system. Preferably, the cell vial would be bar coded so that a bar code reader could establish the identity of the vial and then match this information in a pre-established database regarding the contents such as cells, operator, type of microcarrier, and growth conditions. The vial could also be equipped with a radio frequency identification chip (RFID) or other means of labeling. The vial would be placed into an input device in the form of a window, port, or orifice. A mechanical assembly would acquire the vial and transfer the vial to a device that would warm the vial to 37° C. The warming device would be configured to perform a controlled reproducible thawing profile. In addition, the means to sterilize the outside of the vial would be engineered into the system, such as (but not limited to) bathing the vial in ethanol, isopropanol, bleach, hydrogen peroxide, or exposing to a gas plasma. Following the controlled thaw and vial sterilization, the vial cap would be removed, or the vial would be pierced with a pipette on the pipetting effector of the robot under sterile conditions. The contents would be aspirated by a pipette and then transferred to the sterile culture vessel. Microcarriers, media, and growth factors would be introduced into the culture vessel while in the controlled growth environment (incubator), or prior to placing the culture vessel into the incubator. A mixture of microcarriers and cells are allowed to rest for at least one hour in media in the culture vessel so that cells may become attached to the surface of the microcarrier. The length of time allowed for the cells to attach to the microcarriers will depend on the type of cell being cultured. Once the cells have attached to the microcarriers, any of the plurality of physical forces previously described to stir or move microcarriers within the cell culture media can be employed.

While cells are growing, a plurality of methods for monitoring cell growth may optionally be employed. Various methods have been tested that demonstrate their use for determining what percentage of the microcarrier surface (on average) is covered with growing cells. For example, one might employ (but not be limited to) any number of conventional analysis, such as spectroscopy in any wavelength of the electromagnetic spectrum, right angle light scatter, image analysis, measuring cell autofluorescence, Raman spectroscopy, mass spectroscopy, protein expression, ability to take up or exclude vital dyes, thymidine uptake, and other means for measuring cell growth. Once cells have reached confluence, or have been arrested in any state of growth, one can then monitor cell health and status using techniques such as (but not limited to) ion transport, intracellular pH, and calcium uptake.

Once cells have reached their desired state of confluence or growth, they may be harvested for a variety of uses including drug discovery, research, and cell product production. Alternatively, the cells may be used as protein or cell product factories, and the media may be harvested by the automated pipettes or pumps. Microcarriers may be harvested by a variety of means after the stirring means is discontinued. Stirring in this context, does not imply a circular motion, but any motion that maintains the microcarriers in suspension. Microcarriers may be harvested from either the bottom of the culture vessel or the top of the culture vessel depending whether one uses sinking or buoyant microcarriers, respectively. Conversely, the media may be harvested from the top or the bottom of the cell culture system depending if the microcarriers are at the top or the bottom. One would normally want to harvest media in the absence of microcarriers, or harvest microcarriers in a minimal amount of media.

Cell assays on harvested microcarriers can be used directly in the various product production processes or bioassays. Alternatively, as explained above, microcarriers may be dissociated using chemical or enzymatic means. In the case of calcium alginate, the microcarriers will spontaneously dissolve in the presence of a low Ca²⁺ medium. The automation system is equipped with the mechanical systems necessary to harvest cells or cell products and transport them directly into the next process. This feature will obviate the need for laboratory technologist labor, as well as reduce the potential for contamination of the cells.

Alternatively, cells may be harvested for long term storage by freezing at −150° C., either directly on the microcarriers or after having been dissociated from the microcarriers. The automated system may be programmed to perform the controlled freezing protocol by using an automated cooling device. Once the cells have been frozen, according to standard freezing protocols, then the cells may be stored for a short period of time (one month or less) in a −80° C. freezer (TechCell, Hopkinton, Mass.) or in a −150° C. freezer, or directly in a liquid nitrogen freezer. The use of a freezer may be obviated by dehydrating the microcarriers containing cells to a state that supports the suspended animation of the cells.

### Uses of Cells on Microcarriers

Orothobiologics is the field of growing structural tissues for replacement or repair. Functionalized and/or engineered microcarriers of the present invention can be used to support the growth and differentiation of cells intended for autologous or heterologous transplantation in plants, animals, or humans. Implant tissue should support the growth of cells on a matrix that may ultimately be absorbed and replaced by the body's own support matrix. Various cells can be grown for use in living beings. In humans, commercially viable replacement cells include chondrocytes (cartilage cells), oesteocytes (bone cells), oesteoblasts, chondrogenic cells, pluripotential cells and mucosal cells for tissue replacement and/or coverage.

The microcarrier culture technologies of the present invention (engineered microcarriers, bioreactor, and automation platform) provides a better source of cells for tissue replacement in humans and conventionally grown cells. Cells produced in the engineered microcarriers of the present invention will enable more rapid production of cells, less damage due to shear stress and impeller collisions, an ability to monitor cell growth and optimize growth conditions in real time, and initiate and maintain the culture in a fully automated and sterile environment. Furthermore, when human, animal, or plant cells are grown on engineered microcarriers, they can be injected directly into tissue for repair or replacement of cells. In this case, paramagnetic particles that have been approved by the FDA for implantation in humans would be used. Alternatively, a strong magnetic field is used to strip the paramagnetic particles from the microcarriers prior to injection. The glass bubbles are biologically inert, however, the use of gas bubbles would be preferable for injectable microcarriers. The ability of engineered microcarriers to be kinetically manipulated allows formation of microcarrier aggregates, which may have better in-vivo viability, or to manipulate microcarriers once they have been placed in the living being.

The following additional embodiments that have not already been disclosed above are here below provided to describe the present invention within the scope of the disclosure:

Microcarriers with Inherent Physical Properties
1. A method to create microcarriers of various shapes and composition which have inherent properties that allow them to respond to external forces [for example, but not limited to microcarriers have inherent dipole moments so they will respond to an electrical and/or magnetic field, inherent compressibility and buoyancy so they will respond to changes in pressure, and inherent autofluorescence that will yield a useful signal when measured with the right device].
2. The method of 1, wherein at least one subpopulation of microcarrier can be any or all of the following: spherical, triangular, trapezoidal, cubic, extended cylinder, hollow, hollow with access openings, tubular (sealed or with an opening at either end or anywhere along its length), porous, or planar shape. Any position along the surfaces of the plurality of shapes that come in direct contact with cell culture media may be chemically modified to allow or disallow cell attachment.
2b. The method of 1 and 2 above, wherein said microcarriers is characterized by a surface that will support the growth of cells.
2c. The method of 1-3 above wherein said microcarriers are characterized by an absence of specific sites capable of supporting the growth of cells.
2d. The method of 1 and 2 wherein said micro-spheres have a mean diameter between 1 nm and 1 mm.
2e. The method of claim 1-2c wherein said microcarriers have a mean diameter between 100 nm and 500 um.
2f. The method of 1-2 wherein the microcarriers have a density of the carrier for cell culture is within the range of 0.8 to 1.4 g/cm., which makes it possible to suspend the microcarriers for cell culture in a culture solution.
2g. The method of 1 above wherein the microcarriers are created by spray coalescence or emulsion polymerization.
3. A breakable biocompatible microcarrier as in 1 or 2 directly above, allowing the cells to be delivered to a site of interest and then the bubble would be broken, collapsed or dissolved by a variety of means. For example, but not a limiting application, one could break the bubbles by delivering ultrasound energy to the same location as the bubbles either in-vitro or in-vivo.
4. A microcarrier with a modified surface to allow attachment of non-anchor dependent cells.

### Engineered Microcarriers

5. A method to impart a detector molecule within or on the microcarrier to measure cell growth and/or activity in living cells growing on or in the microcarrier.
6. A detector molecule within or on the microcarrier which amplifies the signal emitted by another detector molecule in or on the microcarrier as in 4 above.
7. A microcarrier designed for the growth and/or maintenance of anchorage dependent cells incorporating materials which imparts a magnetic dipole or wherein the microcarrier is magnetic containing iron or oxides of iron, or paramagnetic, or wherein the microcarrier has a combination of these features.
8. A microcarrier designed for the growth and/or maintenance of anchorage dependent cells manufactured with materials which impart an ability to control the microcarrier density and/or buoyancy, or contains materials that allow the density or buoyancy of the microcarrier to be controlled by outside forces.
9. A microcarrier described in any claims designed for the growth and/or maintenance of anchorage dependent cells incorporating materials which imparts transparency, and a low autofluorescence relative to the autofluorescence inherent in the cells of interest.

### Applications of Engineered Microcarriers

10. Microcarriers that have been engineered as analytical tools that mimic biological processes.
11. Microcarriers as in 8 above that have been engineered to monitor and measure cell migration, invasion, and metastasis.
12. Microcarriers as in any of 7 and 8 above that are engineered to mimic the biological activities of various organs including but not limited to the blood brain barrier, intestinal track, kidney, liver, heart, lungs, bone marrow, skin, and blood vessels.
13. Microcarriers are used as an attachment surface for stem cells that are derived from a variety of sources such as (but not limited to) adipose tissue, embryos, and peripheral circulation. The simulated microgravity environment is favorable for promoting the maintenance or differentiation of stem cells into progeny cells.

### Combinations of Physical Properties

14. Microcarriers which have a combination of properties in any or all of claims 1-6.

### Kinetic Energy

15. A method to control the kinetic energy parameters; acceleration, movement, velocity of movement, absolute position, and rotational speed of a microcarrier in a liquid.
16. A method to control the kinetic energy parameters within a microcarrier in a liquid.
17. A method to control the kinetic energy parameters as in 1 and 2 above in clusters of microcarriers in a liquid.

### Controlling Each Physical Force Impinging on the Microcarriers

18. A method to control the magnetic forces that impinge on microcarriers as in any or all of 1-7 above.
19. A method to control buoyancy or kinetic energy of microcarriers as in any or all of 1-7 above by controlling the external pressure on the liquid containing the microcarriers.
20. A method to control the kinetic energy parameters of any microcarrier as well as microcarriers as in any or all of 1-7 above by inducing a thermal gradient.

### Controlling Many Physical Forces

21. Microcarriers according to any of the previous 1-7 above, which contain substances indicating their orientation and/or direction of travel.
21b. Microcarriers as in any claim 1-7 and/or 21 that participate in a feedback loop where their kinetic energy and or direction of travel and or orientation can be controlled external to the culture vessel based on their orientation as determined by a method described in 21 above.

### Measuring Microcarrier Orientation and Cell Biochemistry and Physiology

22. A method for examination of microcarriers as in any of 1-7 above to determine orientation, cell growth, and cell health.
22b. The method of 1 above wherein at least one sub-population of microcarriers has a luminescent, fluorescent, or colorimetric property and wherein signals emitted by said microcarriers can be detected by any method that includes: (a) whole frame imaging; (b) partial frame imaging, and (c) signal capture as a static recording or signal measurement or time based recording or signal measurement.
23. A method as in 15 above using any device measuring changes in the electromagnetic spectrum emitted by cells on or in microcarriers, including (but not limited to) a spectrophotometer, fluorometer, Raman light scattering instrument, luminometer, fluorescence polarimiter, and/or light scatter instrument.
24. A method to detect cellular biochemical signals given off by the microcarrier [for example; examining microcarriers in a solution to determine drug absorption].

### Bioreactor

24b. A bioreactor that contains the microcarrier and media in which it grows.
25. A bioreactor for optimizing the growth of cells on microcarriers consisting of a unit containing a vessel to hold cell culture media, a device to supply heat to the microcarrier culture to maintain optimal growth and maintenance temperature, a device to supply a constant supply of gas (both CO₂, air, and/or oxygen), an external control device to control kinetic energy; position, orientation, and movement of the microcarriers as in 8-13 above, and devices to maintain sterility within the bioreactor.
26. A bioreactor as in 18 above, but constructed in a modular fashion so that multiple bioreactors can be used simultaneously and share the same sources for energy, gases, and/or external control device, and can allow the media and microcarriers to remain sterile.
27. A bioreactor as in 18 and 19 above employing a vessel that allows ample oxygenation of the cell culture media through the walls of the vessel, for example, a polyfluorinated bag, but does not allow for appreciable loss of moisture or the transmission of virus or bacteria.
28. A bioreactor as in 18-20 above which employs an external computing device to control the flow of gases, temperature, humidity, sterility.

### Automation

29. An automated cell culture system consisting of a single or plurality of bioreactors as in 18-21 above.
30. An automated cell culture system as in 21 above incorporating a device to add media to and withdraw media from the bioreactors.
31. An automated cell culture system as in any of the 21-22 above incorporating a device to accept the input of a vial of cells for culture.
32. An automated device that sterilizes the vial of cells provided to the automated cell culture device prior to thawing the cells, opening the container and transferring the thawed cells to a bioreactor as in any 18-21 above containing cell culture media.
33. An automated device that maintains, grows, and monitors the progress of cultured cells including maintaining sterility, changing media, maintains optimal kinetic energy associated with the microcarriers, and harvests cells at an appropriate time.
34. An automated device, as in 26 above, containing a computer system that monitors and adjusts the performance of the automated system based on any of the above 21-25 based on the cell culture needs.
35. An automated device that prepares and freezes cells for long term storage that uses a controlled freeze profile for lowering the temperature of the cells to be frozen while they are still attached to the microcarrier.
36. An automated device that prepares and freezes cells as in 27 above, but employs strong magnetic field to prevent microcrystallization of ice within the cell or microcarrier.
37. An automated device that prepares cells for long term storage but employs desiccation of the cell/microcarrier complex.
38. An automated system that contains the hardware and reagents controlled by a software algorithm necessary to produce microcarriers on demand.

Although the invention has been described in detail for the purposes of illustration, it is to be understood that such detail is solely for that purpose and that variations can be made therein by those skilled in the art without departing from the spirit and scope of the invention.

All cited references are herein incorporated in their entirety by reference.

### REFERENCES

1. Felder R A, Gildea J. Automated cell culture system and process. U.S. Patent 60/488,068, Provisional 07.17.03.
2. Mizrahi A. Biologicals produced from animal cells in culture—an overview. Biotechnol Adv. 1988; 6(2):207-20.
3. Talbot P, Keen M J. Utilization of DEAE-cellulose as a microcarrier material. Dev Biol Stand 1980; 46:147-149.
4. Johansson A, Nielsen V. Biosilon a new microcarrier. Dev Biol Stand 1980; 46:125-129.
5. Wissemann K W, Jacobson B S. Pure gelatin microcarriers: synthesis and use in cell attachment and growth of fibroblast and endothelial cells. In-Vitro Cell Devl Biol 1985; 21:391-401.
6. Huang Y M, Rorrer G L. Cultivation of microplantlets derived from the marine red alga Agardhiella subulata in a stirred tank photobioreactor. Biotechnol Prog March/April 2003; 19(2):418-27.
7. Kwon J Y, Peng C. Calcium-alginate gel bead cross-linked with gelatin as microcarrier for anchorage-dependent cell culture. Biotechniques 2002; 33:212-218.
8. Thilly W G, Levine D W. Microcarrier culture: a homogeneous environment for studies of cellular biochemistry. Methods Enzymol. 1979; 58:184-194.
9. Van Wezel A L. Growth of cell-strains and primary cells on micro-carriers in homogeneous culture. Nature 1967; 216:64-65.
10. Koichi K, Umezawa K, Funeriu D P, Myake M, Miyake J, Nagamune T. Immobilized culture of non-adherent cells on an oleyl poly(ethylene glycol) ether-modified surface. BioTechniques, 2003; 35(5):1014-1021.
11. Zhang S L, Zhang S L, Yan L, Altman M, Lassle M, Nugent H, Frankel F, Lauffenburger D A, Whitesides G M, Rich A. Biological surface engineering; a simple system for cell pattern formation. Biomaterials 1999; 20; 1213-1220.
12. Chen C S, Mrksich M, Huang S, Whitesides G M, Ingher D E. Geometric control of cell life and death. Science 1997; 276:1425-1428.
13. Chen C S, Mrksich M, Huang S, Whitesides G M, Ingher D E. Micropatterned surfaces for control of cell shape, position, and function. Biotechnol Prog 1998; 14:356-363.
14. Ostuni E, Yan L, Whitesides G M. The interaction of proteins and cells with self-assembled monolayers of alkane-thiolates on gold and silver. Colloids Surf. B Biointerfaces 1999; 15:3-30.
15. Ziauddin J, Sabatini D M. Microarrays of cells expressing defined cDNAs. Nature May 2001; 411(6833):107-10.
16. Hillegas W J, Varani J, Helmreich D L. Collagen-coated polystyrene microcarrier beads, U.S. Pat. No. 4,994,388, Feb. 19, 1991.
17. Klock G, Frank H, Houben R, Zekorn T, Horcher A, Siebers U, Wohrle M, Federlin K, Zimmermann U. Production of purified alginate suitable for use in immunoisolated transplantation. Appl Microbiol Biotechnol 1994; 40:638-643.
18. King G, Daugulis A, Faulkner P, Goosen M. Alginate-polylysine microcapsules of controlled membrane molecular weight cutoff for mammalian cell culture engineering. Biotechnol Prog 1987; 3:231.
19. Strand B L, Morch Y A, Skjak-Braek G. Alginate as immobilization matrix for cells. Minerva Biotecnologica 2001; 12:223-233.
20. Thomas N, Andersson U. Microfabricated apparatus for cell based assays. US Patent Publication #2004/0058408 A1, Mar. 25, 2004.
21. Mundt W. Method for releasing cell cultures from microcarriers. U.S. Pat. No. 5,100,799, Mar. 31, 1992.
22. Farghali H, Kamenikova L, Hynie S. The concept of application of immobilized and perfused mammalian cells (a bioreactor model) in biomedical research. Physiological Research, 1994; 43(2):117.
23. Hu W S, Aunins J G. Large-scale mammalian cell culture. Current Opinion in Biotechnology, 1997; 8:148.
24. Chu L, Robinson D K. Industrial choices for protein production by large-scale cell culture. Current Opinion in Biotechnology 2001; 12:180.
25. Meisenholder G. Postmodern culture: maximizing cell culture output at every level. The Scientist 1999; 13(14):21.
26. Grandics P, Szathmary S, Szathmary Z, O'Neill T. Integration of cell culture with continuous, on-line sterile downstream processing. Annals of the New York Academy of Sciences 1991; 646:322.
27. Davis J M, Hanak J A. Hollow-fiber cell culture. Methods in molecular biology 1997; 75:77-8.
28. Kurkela R, Fraune E, Vihko P. Pilot-scale production of murine monoclonal antibodies in agitated, ceramic-matrix or hollow-fiber cell culture systems. Biotechniques 1999; 15(4):674.
29. Schwarz R P, Wolf D A. Rotating bioreactor cell culture apparatus. U.S. Pat. No. 4,988,623, Jan. 29, 1991.
30. Schwarz R P, et al. Cell culture for three-dimensional modeling in rotating-wall vessels: An application of microgravity. Journal of Tissue Culture Methods 1992; 14:51-8.
31. Shuler M L, Babis J G, Sweeney L M, Johnson B E. Automated, multicompartmental cell culture system. U.S. Pat. No. 5,612,188, Mar. 18, 1997.
32. Wolf D A, Goodwin T J. Growth stimulation of biological cells and tissue by electromagnetic fields and uses thereof. U.S. Pat. No. 6,673,597, Jan. 6, 2004.
33. Jovanovic G N, Pinto-Espinoza J, Sornchamni T, Reed B, Wheeler R R Jr., Atwater J E, Akse J R. Development of enabling technologies for magnetically assisted gasification of solid wastes. SAE Technical Paper 2003-01-2374, presented 33rd International Conference on Environmental Systems, Vancouver, BC, Jul. 7-10, 2003.
34. Kempner M E, Felder R A. A review of cell culture automation. JALA 2002; 7(2):56-62.
35. Lütkemeyer D, Poggendorf I, Sherer T, Zhang J, Knoll A, Lehmann J. First steps in robot automation of sampling and sample management during cultivation of mammalian cells in pilot scale. Biotechnology Progress. 2000; 16:822.
36. Selznick S H, Thatcher M L, Brown K S, Haussler C A. Development and application of computer software for cell culture laboratory management. In-vitro Cellular and Developmental Biology—Animal 2001; 37(1): 55.
37. Hu W S, Piret J M. Mammalian cell-culture processes. Current Opinion in Biotechnology 1992; 3(2):110.

1. An engineered microcarrier suitable for the automated growing of cells comprising a hydrogel polymer capable of providing a substrate that will support the growth of cells in culture,
wherein said hydrogel polymer further comprises at least one paramagnetic particle which renders the microcarrier responsive to at least one physical force,
and wherein the microcarrier has a diameter of about 1 nm to 1 mm, a density of about 0.8 to 1.4 g/cm³, optical clarity, has low autofluorescence relative to the autofluorescence inherent in the cells, is dissolvable without the use of an enzyme, and has a surface coating that will promote or enhance cell adhesion.

2. The engineered microcarrier of claim 1 wherein said hydrogel composition is selected from the group consisting of alginate, gelatin, polyacrylamide-copolymerized with collagen or gelatin, polyacrylamide with modified charge, alginate copolymerized with gelatin and a combination thereof.

3. The engineered microcarrier of claim 1, wherein said material imparts an ability to control the microcarrier density and/or buoyancy, or allows the density or buoyancy of the microcarrier to be controlled by at least one physical force.

4. (canceled)

5. The engineered microcarrier of claim 1, wherein said cells are human, mammalian, animal or plant cells.

6. The engineered microcarrier of claim 1, wherein said physical force comprises electromagnetic energy.

7. The engineered microcarrier of claim 1, wherein the shape of said microcarrier is spherical, triangular, trapezoidal, cubic, extended cylinder, hollow, hollow with access openings, tubular sealed at the ends, tubular with an opening at either end, tubular with at least one opening along its length, porous, or planar shape.

8. The engineered microcarrier of claim 7, wherein any position along the surfaces of any one of the plurality of shapes that come in direct contact with cell media may be chemically modified to allow or disallow cell attachment.

9. (canceled)

10. The engineered microcarrier of claim 1, wherein said microcarrier has a mean diameter between approximately 100 nm and 500 μm.

11. (canceled)

12. The engineered microcarrier of claim 1, wherein said microcarrier further comprises a detector molecule within or on the microcarrier to measure cell growth and/or activity in said cells growing in culture on or in the microcarrier.

13. The engineered microcarrier of claim 1, wherein said detector molecule amplifies the signal emitted by another detector molecule in or on the microcarrier.

14. The engineered microcarrier of claim 1, wherein said microcarrier further comprises a ligand or reporter that reports a stimulus and/or response to a stimulus and is covalently or non-covalently linked to the surface and/or interior of the microcarrier.

15. The engineered microcarrier of claim 14, wherein said reporter is a fluorescent or bioluminescent molecule.

16. A functionalized microcarrier suitable for growing cells comprising the engineered microcarrier of claim 1, wherein said microcarrier further comprises at least one ligand or reporter that reports a stimulus and/or response to a stimulus and is covalently or non-covalently linked directly or indirectly through a functional group on the surface and/or interior of the microcarrier.

17. The functionalized microcarrier of claim 16, wherein said reporter is a fluorescent or bioluminescent molecule.

18. A bioreactor suitable for growing cells comprising:
(a) a culture vessel comprising at least one engineered microcarrier of claim 1 comprising at least one cell and culture medium sufficient for growth of said cell; and
(b) at least one source for generating at least one physical force to which said microcarrier is responsive.

19. The bioreactor of claim 18, wherein said culture vessel is a polyfluorinated bag.

20. The bioreactor of claim 18, wherein said hydrogel composition is selected from the group consisting of alginate, gelatin, polyacrylamide-copolymerized with collagen or gelatin, polyacrylamide with modified charge, alginate copolymerized with gelatin and a combination thereof.

21. The bioreactor of claim 18, wherein said material of said hydrogel composition imparts an ability to control the microcarrier density and/or buoyancy, or allows the density or buoyancy of the microcarrier to be controlled by at least one physical force.

22. The bioreactor of claim 18, wherein said material of said hydrogel composition imparts a magnetic dipole, is a magnetic particle, a paramagnetic particle, an air bubble, a gas bubble, a hollow bead or a combination thereof.

23. The engineered bioreactor of claim 18, wherein said physical force comprises electromagnetic energy, sonic energy, thermal energy, pressure, gravity or a combination thereof.

24. An automated bioreactor suitable for growing cells comprising:
(a) at least one bioreactor that comprises:
(1) a culture vessel comprising at least one engineered microcarrier of claim 1 comprising at least one cell and culture medium sufficient for growth of said cell; and
(2) at least one source for generating at least one physical force to which said microcarrier is responsive; and
(b) at least one control system that controls the function of the bioreactor and the generation of the physical force to control said microcarrier.

25. The bioreactor of claim 24, wherein said hydrogel composition is selected from the group consisting of alginate, gelatin, polyacrylamide-copolymerized with collagen or gelatin, polyacrylamide with modified charge, alginate copolymerized with gelatin and a combination thereof.

26. The bioreactor of claim 24, wherein said material of said hydrogel composition imparts an ability to control the microcarrier density and/or buoyancy, or allows the density or buoyancy of the microcarrier to be controlled by at least one physical force.

27. The bioreactor of claim 24, wherein said material of said hydrogel composition imparts a magnetic dipole, is a magnetic particle, a paramagnetic particle, an air bubble, a gas bubble, a hollow bead or a combination thereof.

28. The bioreactor of claim 24, wherein said cells are human, mammalian, animal or plant cells.

29. The bioreactor of claim 24, wherein said physical force comprises electromagnetic energy, sonic energy, thermal energy, pressure, gravity or a combination thereof.

30. The bioreactor of claim 24, wherein said microcarrier further comprises a detector molecule within or on the microcarrier to measure cell growth and/or activity in said cells growing in culture on or in the microcarrier.

31. The bioreactor of claim 30, further comprising a monitoring system to detect said detector molecule.

32. The bioreactor of claim 24, further comprising an assay system to analyze the cells contained on the microcarriers and cell products thereof.

33. The bioreactor of claim 33, wherein said assay system is directly connected to said culture vessel through a closable opening.

34. The bioreactor of claim 24, further comprising a microcarrier manufacturing system to produce the microcarriers.

35. The bioreactor of claim 34, wherein said microcarrier manufacturing system is directly connected to said culture vessel through a closable opening.

36. The bioreactor of claim 34, further comprising a monitoring system to detect a reporter molecule associated with said microcarrier, an assay system to analyze the cells contained on the microcarriers and cell products thereof and a microcarrier manufacturing system to produce the microcarriers.

37. An automated bioreactor system comprising more than one automated bioreactors of claim 24.

38. The bioreactor system of claim 37, wherein said system comprises a single control system that controls the function of each one of said bioreactors and the generation or control of the physical force to control said microcarrier.

39. An automated bioreactor system comprising more than one automated bioreactors of claim 36.

40. A method of growing cells comprising:
(a) adding microcarriers of claim 1 to culture media in a bioreactor;
(b) applying physical forces or allowing gravity to put the cells and microcarriers together;
(c) allowing said microcarriers to remain in contact with living cells until the living cells attach to said microcarriers;
(d) applying physical forces to impart kinetic energy to said microcarriers containing attached cells as in (c);
(e) applying physical forces to move microcarriers to allow the change of expended culture media with fresh media using manual or automated methods;
(f) applying physical forces to move microcarriers to allow them to be harvested to passage cells to new cultures as in (a)-(e); and/or
(f) applying physical forces to move the microcarriers to a method to harvest said microcarriers and transfer them to another culture vessel or into an assay system.

41. A method of growing cells in suspension comprising:
(a) adding microcarriers that disallow cell attachment as in claim 8 to culture media;
(b) applying physical forces or allowing gravity to impart kinetic energy to the culture media;
(c) applying physical forces to move microcarriers and cells to allow the change of expended culture media with fresh media using manual or automated methods;
(d) applying physical forces to move microcarriers and cells to allow the cells to be harvested to passage cells to new cultures as in claim (a)-(c); and
(e) applying physical forces to move the microcarriers to a method to harvest said cells and transfer them to another vessel or into an assay method.

42. A method of storing cells on or in microcarriers of claim 1, cultured in a bioreactor by freezing or dehydrating said microcarriers containing cells grown in culture on said microcarriers.

43. The method of re-culturing said stored cells as in claim 42 by thawing or rehydrating and culturing as in a cell culturing system.

44. A method of storing cells cultured in a bioreactor with microcarriers as in claim 8 by freezing or dehydrating said microcarriers containing cells grown in culture on said microcarriers.

45. A method of re-culturing said stored cells as in claim 44, by thawing or rehydrating and culturing as in a cell culturing system.

46. The engineered microcarrier of claim 1, wherein said cells are human and the microcarrier has a spherical shape.
